(19) United States
(12) Patent Application Publication
(10) Pub. No.: US 2002/0058250 A1
(43) Pub. Date: May 16, 2002

(54) EXTRACTION AND UTILISATION OF VNTR ALLELES

(75) Inventor: GREG FIRTH, HITCHIN, HERTS (GB)

Correspondence Address: MARSHALL O'TOOLE GERSTEIN, MURRAY & BORUN, 6300 SEARS TOWER, 233 SOUTH WACKER DRIVE, CHICAGO, IL, 606066402

(73) Assignee: Marshall, Gerstein & Borun

(21) Appl. No.: 09/380,932

(22) PCT Filed: Mar. 20, 1998
(86) PCT No.: PCT/GB98/00840

(30) Foreign Application Priority Data
Mar. 21, 1998 (EP) 98/42867

(51) Int. Cl. C12Q 1/68; C07H 21/04; C12P 19/34; C12N 5/06; C12N 5/04; C12N 15/74

(52) U.S. Cl. 435/6; 435/91.1; 435/325; 435/410; 435/320.1; 536/23.1; 536/24.1; 536/24.3; 536/24.31; 536/24.33; 536/25.32; 536/25.5; 536/25.6; 435/243

(57) ABSTRACT

The invention presented is a novel method for the extraction of VNTR alleles and for the concomitant detection of polymorphic markers for inherited traits at multiple loci by simultaneous comparison of complex genomes from multiple individuals. The product is designated a Total Representation of Alleles that are Informative for a Trait (TRAIT). These alleles may be used directly as genetic markers or may be used as vehicles to facilitate precise localisation of sequence variations responsible.

### GLOSSARY OF TERMS AND ABBREVIATIONS

adapter nucleotide sequences, usually comprising annealed complementary oligonucleotides, ligated to DNA fragments that allow specific amplification and manipulation of those fragments

AFLP amplified fragment length polymorphism

allele one of several possible alternative sequence variations at any one locus

amplimer the product, or pool of products, generated by amplification with the adapter primer and an ‘internal primer’

DNA deoxyribonucleic acid

DNA fingerprint the display of a set of DNA fragments from a specific DNA sample

GMS genomic mis-match scanning

individual a member of any species subject to investigation

heteroduplex a duplex of two alleles derived from different individuals, sets of individuals or populations

heterozygygous alleles at the same locus of each of the paired chromosomes in a diploid cell being different homoduplex a duplex of alleles derived from the same individual, set of individuals or population

homozygous alleles at the same locus of the paired chromosomes of a diploid cell being identical

locus a specific position on a chromosome

mis-match one or more bases in a duplex that fail to form stable hydrogen bonds with opposing bases

NASBA nucleic acid sequence based amplification

PCR pojymerase chain reaction

RAPD random-amplified DNA markers

RDA representational difference analysis

RFLP restriction fragment length polymorphism

trait a distinguishing feature or characteristic manifesting itself physically, chemically or biologically

TRAIT Total Representation of Alleles that are Informative for a Trait

VNTR variable number tandem repeat, also referred to as simple sequence repeats (encompassing all repeats of two or more nucleotides that may be continuous or interrupted by short non-repetitive sequence, including minisatellites and microsatellites).

### FIELD OF THE INVENTION

The field of this invention is the detection of polymorphic variation in complex genomes, which is the mainstay of the study of hereditary traits in all organisms. Since polygenic traits far outweigh those that are monogenic, a procedure that allows the isolation in concert of several informative polymorphisms within the complex genomes of multiple individuals would provide an extremely powerful tool for the investigation of hereditary traits.

The invention differs fundamentally from all other techniques that have been previously employed by:

(i) permitting mass generation of VNTRs quickly and easily from DNA

(ii) generating polymorphisms that are both linked and informative for a trait;

(iii) reproducing and preserving the polymorphic allele, as it occurs in the genome;

(iv) negating problems that are features of other polymerase chain reaction based techniques; including miss priming, reaction contamination and generation of spurious products;

(v) negating the need for investigations to be confined to families of closely-related individuals;

(vi) permitting the analysis of polygenic traits;

(vii) having a sparing requirement for DNA starting material.

The invention therefore represents a major advancement in the ability of workers in the biomedical fields to screen simple or complex genomes, rapidly and with fidelity, for polymorphisms co-segregating with advantageous or deleterious monogenic or polygenic hereditary traits. There is enormous potential for advancement of medicine, veterinary medicine, forensic science, agriculture, animal husbandry and biotechnology, by the generation of polymorphic markers co-segregating with hereditary disease or traits of social or economic importance. The invention will also serve to facilitate mutation analysis for all relevant organisms.

### Introduction

DNA is a double stranded linear polymer composed of repetitions of four mononucleotide units. The sequence in which these units are arranged gives rise to a genetic code, referred to as the genome. Although the genomes of all individuals within a species are essentially homologous, subtle variations exist which impart individuality. Locations of the genome at which more than one sequence variation may exist are termed polymorphisms, each variant of that sequence representing an allele. Polymorphisms in gamete-forming germinal cells will be inherited by subsequent generations of progeny. By studying the combination of polymorphisms in the genome of an individual a unique code (‘fingerprint’) can be assigned and the ancestry of that individual can be determined. Furthermore, a polymorphism found to be linked and co-segregating with a particular genetic trait or hereditary disease may be used as a marker for genetic screening of that trait or disease in other individuals.

The study of advantageous or deleterious hereditary traits in complex genomes has been the subject of considerable interest due to its economical, medical and social implications. The establishment of protocols that allow the comparison of nucleic acid sequences in complex genomes and the isolation of differences unique to a subset of those sequences is a fundamental requirement of this field of study.

A number of protocols have been used in animals and plants for the comparison of nucleic acid sequences and isolation of differences between those sequences in individuals. These protocols involve restriction fragment length polymorphism (RFLP), random-amplified polymorphic DNA markers (RAPD), amplified fragment length polymorphism (AFLP), representational difference analysis (RDA), genomic mis-match scanning (GMS), and linkage analysis of variable number tandem repeats (VNTR). These protocols detect polymorphisms by assaying subsets of the total DNA sequence variation in a genome. Polymorphisms detected by RFLP, AFLP, and RDA rely on the generation of a fingerprint ladder by gel-electrophoresis which reflects restriction fragment size variation. RAPD polymorphisms result from sequence variation at primer binding sites and differences in length between primer binding sites. GMS polymorphisms result from sequence variation within heterohybrid molecules comprising restriction fragments derived from two related individuals. Linkage analysis involves the detection of length variation of variable number tandem repeats (VNTRs) and co-segregation of one allele with a trait of interest.

### RFLP

RFLP analysis relies on the cleavage of a nucleic acid sequence by restriction endonucleases and separation of the resulting fragments by gel electrophoresis. The fragments are blotted onto a membrane and hybridized to labelled probes to allow detection of fragment length variation. This technique may be of use in the study of a single isolated locus or gene fragment, but where an investigation is not confined to an isolated sequence it is inadequate. Further limitations are that only a small number of the polymorphisms generated may be informative, there is a high demand for DNA starting material, and the method is labour intensive.

### RAPD

RAPD is a commonly used PCR-based polymorphic marker technique in genomic fingerprinting and diversity studies, particularly for plant species. This technique involves the use of a single ‘arbitrary primer’ which gives rise to amplification of regions of genome where there is sufficient homology between the sequences of genomic DNA, in the 5′ to 3′ direction, and that of the arbitrary primer. The amplified products are separated by gel electrophoresis. Subtle variations of this method include arbitrary primed-PCR (AP-PCR) and DNA amplification fingerprinting (DAF). However, the principle of arbitrary priming and amplification of DNA by PCR for difference analysis is common to all. Advantages compared to RFLP are that these methods are more rapid, have a lower demand for DNA, and do not require prior knowledge of sequence. A limitation in common with RFLP is that each analysis can only compare the genomes of two individuals. Although several loci can be evaluated concomitantly by this method, detection of polymorphisms requires observation of variation in band patterns by gel-electrophoresis and is subject to errors of superimposition of different alleles of similar electrophoretic mobility. Many bands may be faint and difficult to interpret, and it is difficult to achieve consistent results in repeat experiments. In common with the majority of PCR techniques, the results are prone to error by subtle changes in reaction conditions, reagent contamination, and the generation of inconsistent banding patterns. This lack of reliability limits the usefulness of such techniques in the ‘typing’ of individuals.

### AFLP

AFLP analysis (EP, A, 0534858; Zabeau M et al.) involves restriction endonuclease digestion of DNA and ligation of the generated restriction fragments to adapters. Using primers complementary to the adapter sequence, the restriction fragments are amplified by PCR, and the products are separated by gel-electrophoresis, differences in band patterns revealing polymorphisms. Microsatellite-AFLP (WO 96/22388; Kuiper M et al.) is a modification of this technique in which two or more restriction enzymes, at least one of which cuts at a simple sequence repeat, are used to cleave DNA into fragments that are ligated to adapters. The fragments are amplified with primers complementary to the adapter sequence. In common with RAPD, several loci can be evaluated concomitantly by this method, but detection of polymorphisms requires observation of variation in band patterns by gel-electrophoresis and is subject to errors of superimposition of different alleles of similar electrophoretic mobility. The ability to score bands on an AFLP fingerprint is compromised by generation of large numbers of bands of which some may be very faint and difficult to interpret. Furthermore, the technique is prone to errors that are common to all PCR based techniques, summarised above, and suffers from an inability to analyse multiple complex genomes simultaneously. This is compounded by the generation of bands, by incomplete restriction of the template DNA, that do not reflect true polymorphisms. AFLP and RAPD analyses therefore share many of the same limitations. An additional problem is that AFLPs, rather than being evenly dispersed through out the genome, are reported to be clustered around centromeres. Consequently, this method may not allow the generation of polymorphisms that co-segregate with sequence differences of interest if they are located at a distance from centromeres. This problem is reflected in the reduced rate of polymorphism detection compared to techniques such as linkage analysis. Furthermore, the complexity of the experimental data derived by AFLP becomes exaggerated with increasing complexity of the genome subject to analysis. Consequently, although it has been possible to investigate the genomes of some plant species by AFLP analysis, the relatively complex genomes of higher eukaryotic species may be beyond the useful capacity of this technique.

### RDA

RDA involves restriction endonuclease digestion of DNA, ligation of the fragments to adapters and amplification by PCR. Differences between compared genomes are selected by successive rounds of subtractive hybridization and kinetic enrichment such that regions of difference predominate. This technique is prone to erroneous results through reaction contamination and generation of spurious products. In addition, a fundamental requirement of RDA is the availability of families of closely related individuals, some of which are manifesting the trait of interest. Where RDA is performed on anything other than closely related or highly inbred genomes the multiplicity of differences is too vast for succinct and useful analysis.

### GMS

GMS is technique for mapping regions of identity-by-descent of two related individuals. The entire genome is compared in a single hybridisation that has a high demand for DNA since the genomic samples are not amplified. Freedom from the need of prior map information, conventional markers, or gel electrophoresis are to its advantage. However, the method is restricted to use on the genomes of only two related individuals.

Restriction fragments of the two genomes are hybridised, one of which having been methylated such that heterohybrid molecules can be distinguished through their resistance to digestion by Dpn I and Mbo I that cleave only fully methylated and unmethylated molecules, respectively. Heterohybrids containing homologous strands that lack mismatches are selected and used to probe an array of mapped clones. Although the mis-match proteins used in this technique may resolve point mutations polymorphisms comprising more substantial mismatches that are beyond the limit of this system are not detected. Therefore, in keeping with RFLP, AFLP, RAPD, and RDA, GMS tends to resolve binary polymorphisms that may have low informative power.

In all of the above techniques it is essential that there is a difference in nucleotide sequence at or between primer binding sites or endonuclease restriction sites in order to detect polymorphisms. This highlights the major limitations of these procedures, because in many instances a mutation giving rise to a hereditary trait will not create a sequence difference detectable by variation in primer binding or restriction enzyme digestion. Consequently, a polymorphism linked to a trait of interest will not be identified using these techniques. GMS detects polymorphisms that are incidental to the restriction site and is spared some of the limitations of the other methods. However, in contrast to VNTR polymorphisms, the majority of polymorphisms detected by all of these techniques are not informative.

### Linkage analysis

Linkage analysis is an indirect molecular genetic strategy that involves the systematic comparison of the inheritance of polymorphic VNTRs with the trait of interest in families in which that trait is present. There are a number of types of VNTR, including minisatellites and microsatellites, a feature of all being the repetition of elements of simple sequences. They are polymorphic by virtue of variation in the number of times each element is repeated, giving rise to alleles with variation in length. Since several alternative alleles may exist at any one locus, in contrast to polymorphisms based on variation in primer binding or restriction enzyme digestion, VNTR polymorphic alleles tend to be highly informative. Consequently, where co-segregation of a trait with a particular VNTR allele is demonstrated, the allele may be used as a marker for that trait, or may be used as a vehicle to facilitate identification of the molecular genetic basis of the trait. Microsatellites are ubiquitously distributed throughout all eukaryotic genomes. Consequently, linkage analysis with microsatellites is associated with the highest polymorphism detection rate of the genetic screening methods. Indeed, systematic microsatellite analyses have already been responsible for many advances in the understanding of certain types of common cancer. Linkage analysis therefore has advantages compared to other related methods of difference analysis, the results of which are very reproducible. However, linkage analysis is very time consuming, labour intensive and expensive. Furthermore, since many analyses are performed individually the overall requirement for DNA is extremely high. This is particularly true if a physical map of the genome is unavailable for the selection of informative microsatellites that are evenly distributed throughout the genome. The demonstration of linkage requires the application of elaborate statistical programs and powerful computer software for analysis of the experimental data. This technique is better suited to monogenic defects since the statistical analyses required for multigenic traits are particularly complex. Unfortunately, multifactorial genetic traits are far more prevalent than monogenic defects, making linkage analysis a cumbersome technique for the investigation of the majority of hereditary traits.

The characteristics of an ideal protocol for isolation of polymorphisms co-segregating with disease in complex genomes would include:

(i) the ability to isolate simultaneously and with fidelity the polymorphisms from complex genomes of several individuals

(ii) the ability to isolate several polymorphisms simultaneously, permitting the analysis of polygenic traits

(iii) a high detection rate of polymorphisms that co-segregate with sequence differences in all eukaryotic species, including subtle differences such as those resulting from point mutations

(iv) no requirement for large families of closely related individuals to study traits of interest

(v) no requirement for physical maps of the genome or prior knowledge of genomic sequence

(vi) a requirement for sparing quantities of nucleic acid samples for analysis

(vii) simplicity of use without a need for expensive specialist laboratory equipment or computer software

(viii) potential for widespread application throughout the animal and plant kingdoms

(ix) a robust performance with precision, accuracy and fidelity.

None of the techniques that are currently available fulfil the majority of these ideal characteristics. All are compromised by at least one of several limitations including: expense; lack of speed; requirement for large amounts of DNA; low polymorphism detection rate; an inability to detect small sequence variations such as point mutations; a lack of fidelity with high incidence of artefacts and spurious results; inability to analyse several complex genomes concomitantly; an inability to resolve simultaneously polymorphisms at multiple loci; an intrinsic need for closely related genomes for analysis; a need for prior knowledge of sequence; and complexity of analysis with a need for expensive equipment and computer software. In addition, those techniques that are reliant on large families of closely related individuals are further compromised where there are discrepancies in lineage, so that paternity testing may be an essential preliminary investigation to establish the integrity of each family individual subject to analysis.

### The Invention

The invention is a novel method for generating en masse the VNTRs from genomic or synthetic DNA, while preserving each allele with its flanking sequence. These alleles may be used to produce a ‘fingerprint’ by gel electrophoresis, or they may be used as the starting material in protocols for genotyping individuals or protocols for isolation of polymorphic markers that co-segregate with hereditary traits. The latter may be achieved by mis-match discrimination to yield a pool of alleles that are common to all individuals manifesting a particular trait. Further mis-match discrimination of these selected alleles with the alleles of individuals in which the trait is not present, in solution or fixed to an array, allows purification of VNTRs with alleles that are both linked and informative for the particular trait. The end products, therefore, are designated a Total Representation of Alleles Informative for a Trait (TRAIT).

In one aspect the invention provides a method of making a mixture of VNTR alleles and their flanking regions of the genomic DNA of one or more members of a species of interest, which method comprises the steps of:

a) dividing genomic DNA of the species of interest into fragments,

b) ligating to each end of each fragment an adapter thereby forming a mixture of adapter-terminated fragments in which each 3′-end is blocked to prevent enzymatic chain extension,

c) using a portion of the mixture of adapter-terminated fragments as templates with an adapter primer and a VNTR primer to create a mixture of 5′-flanking VNTR amplimers,

d) using a portion of the mixture of adapter-terminated fragments as templates with an adapter primer and a VNTR antisense primer to create a mixture of 3′-flanking VNTR amplimers,

e) and using genomic DNA of the one or more members of the species of interest as template with the mixture of 5′-flanking VNTR amplimers and the mixture of 3′-flanking VNTR amplimers as primers to make the desired mixture of VNTR alleles and their flanking regions.

The species of interest may be any eukaryotic species from the plant and animal kingdoms. Although they do not show repetitive sequences in quite the same way, prokaryotic species are also envisaged. An individual member of a species may be for example a plant or a micro-organism or an animal such as a mammal.

In another aspect the invention provides a portion of genomic DNA of one or more members of a species of interest, said portion consisting essentially of a representative mixture of alleles of a chosen VNTR sequence and their flanking regions.

The term “representative mixture of alleles” does not necessarily imply that all of the possible alleles, or even most of these possible alleles, of a chosen VNTR sequence are present. Whether a particular allele is present or not, e.g. in the mixture generated by the method defined above, may depend on the nature of a restriction enzyme used in step a) and on other factors.

The invention also provides a portion of genomic DNA of a species of interest, said portion consisting essentially of a representative mixture of 3′-flanking regions of a chosen VNTR sequence, each member of the mixture carrying an adapter at its 3-end, and a representative mixture of 5′-flanking regions of a chosen VNTR sequence, each member of the mixture carrying an adapter at its 5′-end.

The invention also provides a method of treating nucleic acids which consist essentially of a mixture of polymorphic alleles, e.g. of a chosen VNTR sequence and their flanking regions, or alternatively a mixture generated in some other way such as AFLP, microsatellite-AFLP, GMS or RAPD, the mixture being representative of those which manifest a trait of interest, which method comprises separating and then re-annealing strands of the mixture, and separating and discarding any mismatches. Preferably the method comprises the additional step of hybridizing the said mixture with a mixture of corresponding polymorphic alleles, e.g. of the chosen VNTR sequence and their flanking regions, or alternatively a mixture generated in some other way such as AFLP, microsatellite-AFLP, GMS or RAPD, which are representative of those which do not show the trait of interest, and selecting mismatches to provide a mixture of polymorphic alleles which are characteristic of the trait of interest.

The invention also provides kits comprising protocols and reagents for performing the methods herein described.

The salient points of the invention may be represented as follows:

(i) reduction in the complexity of the genome by double positive selection of genomic DNA restriction fragments that both ligate to a chosen adapter and contain a sequence with homology to a chosen primer, employing enrichment of such products by PCR, NASBA or other methods;

(ii) introduction of the selected enriched fragments to a genomic template in such a way that allows recreation of the VNTRs with the flanking sequences within that template, whilst preserving the allele and therefore the informativeness of each locus;

(iii) mis-match discrimination of the generated VNTR alleles to remove any spurious products of amplification that occur through miss priming events, reaction contamination, and subtle variation in reaction conditions;

(iv) selection of only those synthesised VNTRs alleles that are common to all individuals manifesting a particular trait or those alleles that predominate in such a group of individuals. This is achieved by strand dissociation and hybridization, giving rise to mis-match containing heteroduplexes of alleles at any locus that differ among the individuals. These complexes can be rejected by mis-match discrimination. The enriched alleles that are common to individuals manifesting the trait or predominate in that group are sufficiently pure to be used as starting material in other DNA based studies that utilise polymorphic alleles;

(v) rejection of those alleles common to all individuals manifesting a particular trait or predominating in such a group that are also common to individuals in which the trait is not present. This is achieved by strand dissociation and hybridization of the VNTR alleles that are common to individuals manifesting a particular trait of interest or predominating in that group with the VNTR alleles of individuals in which the trait is not present followed by a further round of mis-match discrimination. In this case mis-match containing heteroduplexes and homoduplexes derived from the individuals manifesting the hereditary trait are selected. These represent polymorphic VNTRs with an informative allele that co-segregates with the particular trait of interest. Amplification of these VNTRs from the DNAs of individuals manifesting the trait of interest yields the informative alleles that may be used as DNA markers.

The invention provides a method of selecting genetic elements that are common to one pool of individuals but are absent in a second or present at a lower level. An obvious variation on this theme is the selection of genetic elements that are absent in one pool of individuals but are present in a second by judicious selection, during the course of the procedure, of allele duplexes that are either with or without a mis-match.

For simplicity, the protocol may be considered in three separate sections: generation of VNTR alleles; mis-match discrimination; and selection of alleles informative for a trait. The text is illustrated with a number of diagrams to facilitate description of the invention.

### Generation of VNTR alleles

The protocol describes a method of generating with fidelity the VNTR alleles with their flanking sequences en masse from the genomic DNA of one individual, or the pooled DNAs of several individuals. The initial step involves fragmentation of the genomic DNA physically, chemically or enzymatically, the aim of which is to obtain genomic fragments that contain VNTRs all of which being of an amplifiable length. The use of one or more restriction enzymes gives rise to uniform fragmentation of the genomic sample and constitutes the preferred technique. With judicious choice of restriction enzymes that cut frequently there is potential for generation en masse of every VNTR of the chosen type within a genome or pool of genomes since virtually all fragments will be sufficiently small for efficient amplification. It should be noted that the phenotype of individuals contributing genomic DNA for this fragmentation is unimportant. Indeed, the genomes restricted in this way need not be derived from any individual, or pool of individuals, that have been selected by virtue of their phenotype for investigation of a particular trait of interest.

[chemical structure]

The restriction fragments are ligated to an adapter by which the fragments may be amplified or manipulated. The sequence of the longer oligonucleotide contained within the adapter is chosen such that it fails to generate any products when added as the primer to an amplification reaction containing genomic DNA as template. Termini are introduced physically, chemically or enzymatically to all available 3′ ends to prevent their extension under the influence of a DNA polymerase. They may be introduced in one of several ways including: (A) addition of the terminus prior to ligation; (B) addition of the terminus following ligation; (C) addition of the terminus during ligation. The spectrum of available termini that are suitable for this purpose include, but are not limited to, dideoxynucleotide triphosphates.

(A) A method by which termination may be achieved of all 3′ ends with dideoxynucleotide triphosphates prior to ligation is through the action of a DNA polymerase, including Terminal deoxynucleotidyl transferase, in the presence of a chosen dideoxynucleotide triphosphate.

[chemical structure]

Ligation then follows with an adapter containing an appropriate 5′ recess that accommodates the dideoxynucleotide triphosphate terminus on each strand.

[chemical structure]

(B) A method by which termination may be achieved of all 3′ ends with dideoxynucleotide triphosphates following ligation is through the action of a DNA polymerase in the presence of a chosen dideoxynucleotide triphosphate.

[chemical structure]

(C) A method by which the ligated 3′ ends can achieve termination during the ligation process is through incorporation of a suitable 3′ terminus and a 5′ phosphate on the shorter oligonucleotide during its synthesis such that this oligonucleotide will form a covalent bond with the genomic fragments under the influence of a enzyme such as T4 DNA ligase. Again, suitable termini include but are not limited to dideoxynucleotide phosphates, there being a variety of other modifications and deoxynucleotide analogues that will prevent extension of the 3′ ends under the influence of a DNA polymerase.

[chemical structure]

Of these, method (A) was found to be the most reliable since every genomic fragment that achieves ligation to an adapter is guaranteed to have an appropriate terminus. In addition, it guarantees that inter-fragment ligation is impossible. Method (C) also guarantees that each ligated 3′ end possesses a terminus. However, unlike in the case of method (A), inter-fragment ligation can occur.

Since it is likely that some fragments will contain sites at which one DNA strand is nicked, in order to prevent polymerisation from these sites it is preferable to incorporate into them suitable termini. This may be achieved in a number of ways including, but not limited to, the incubation of the terminated and ligated genomic fragments with a DNA polymerase in the presence of all dideoxynucleotide triphosphates.

The longer oligonucleotide that is contained within the adapter may be used as the adapter primer in amplification reactions containing the genomic fragments that have been appropriately ligated and blocked by addition of termini at all potential sites of polymerisation. However, in the absence of ‘internal’ priming from another nucleotide sequence, the amplification of DNA is impossible. However, if another nucleotide sequence successfully anneals and achieves polymerisation to the limit of the adapter, an adapter primer binding site is created. Binding of the adapter primer will allow polymerisation of DNA to the limit of the annealed nucleotide sequence. If the nucleotide sequence represents a primer, or represents a nucleotide sequence containing a primer binding site, introduction of the adapter primer and the ‘internal primer’ allows specific exponential amplification of products only from those fragments that successfully ligated to the adapter and contain DNA homologous to that of the annealed nucleotide sequence.

If an oligonucleotide with sequence homology to a chosen VNTR is used as the internal primer, only those fragments that have ligated successfully to the adapter and contain the targeted VNTR will be capable of amplification. This gives rise to ‘amplimers’ that flank each VNTR, comprising genomic sequence limited by a restriction site for the chosen restriction enzyme and VNTR sequence with homology to the chosen VNTR primer.

[chemical structure]

A number of different types of VNTR sequence have been identified in a diverse range of species. These include, amongst others, the dinucleotide repeats, trinucleotide repeats and the tetranucleotide lo repeats. Since the (AC)n dinucleotide repeat constitutes the most common VNTR that occurs in the majority of species, primers of appropriate sequence to generate amplimers for this VNTR may be chosen. It can be seen that the introduction of an (AC)n primer will give rise to amplimers that represent one flank of the VNTRs, and introduction of a (GT)n primer will give rise to amplimers that represent the other flank of these VNTRs. However, VNTRs with long repeat lengths will be over represented in the amplimer pool relative to shorter VNTRs by virtue of their greater number of primer binding sites. Similarly, the longer alleles will be over represented relative to the shorter alleles of the same VNTR due to their greater number of primer binding sites. This problem is negated by the introduction of degenerate 3′ ends on the VNTR primers that prevent polymerisation of the annealed primers unless they are aligned with the start of the flanking sequence. The amplification of all VNTRs and all alleles, therefore, will not be biased by their repeat lengths. In the case of (AC)n dinucleotide repeats the following primers may be used:

(AC)nB, where B=C+G+T

(CA)nD, where D=A+G+T

(CA)nD, where D=A+G+T

(GT)nH, where H=A+C+T

(TG)nV, where V=A+C+G

Alternatively, amplimers of other VNTR sequences may be generated in this manner by introduction of the appropriate target-specific primer containing a degenerate 3′ end. Indeed, amplimers constituting genomic sequence that contain or flank any target-specific nucleotide binding site may be generated in the same way.

In the case of (AC)n dinucleotide repeats, the amplimers derived from reactions primed by the (AC)nB and (CA)nD degenerate oligonucleotides may be pooled. An obvious alternative is to generate an amplimer pool by priming amplification reactions with the (AC)nB and (CA)nD degenerate oligonucleotides together. However, this is likely to be less efficient than performing the reactions separately. Similarly, the (GT)nH and (TG)nV primed reactions may be pooled, or reactions containing both of these degenerate primers may be performed. Thus, two amplimer pools may be created, each representing sequences from only one flank of each VNTR.

[chemical structure]

Since only one of the two flanking sequences of all VNTRs is generated in each amplimer pool, the full allele length being absent, the products of amplification are non-informative. However, the full length alleles, together with their flanking sequences, can be recreated with fidelity en masse from genomic DNA by hybridisation of the amplimers to that genomic DNA and subsequent polymerisation of the annealed sequences. As such, the full length ‘affected’ VNTR alleles of individuals manifesting a particular trait of interest may be obtained by hybridisation of the amplimers to the genomic DNAs of those individuals. Similarly, the reciprocal reaction for individuals in which that trait is absent will give rise to the generation of full length ‘wild type’ VNTR alleles and flanking sequences as they occur in the genomes of those individuals. Thus, two pools of VNTRs can be generated containing alleles derived from ‘affected’ DNA and alleles derived from ‘wild type’ DNA. A DNA polymerase that is highly processive is preferred in this application in order to minimise the potential for generation of ‘stutter bands’ that result from strand slippage during polymerisation.

To limit the potential for generation of spurious products by ‘cross-talk’ that occurs through the non-specific association of amplimer strands during hybridisation, it is preferable to remove the VNTR repeat sequences from the amplimers since these repeat sequences will be responsible for the majority of such cross-talk. This may be initiated in a number of ways including, but not limited to, (A) digestion by an enzyme with 3′ to 5′ exonuclease activity; (B) digestion by an enzyme with 5′ to 3′ exonuclease activity; (C) digestion by Uracil DNA glycosylase of an amplimer pool generated with primers containing uracil; (D) digestion by RNase of an amplimer pool generated with an RNA primer.

(A) Providing the 5′ end of the adapter primer has all four nucleotides represented the opposing strand will be similarly endowed. As such, incubation with an enzyme with 3′ to 5′ exonuclease activity, such as T4 DNA polymerase at 12° C. in the presence of only two deoxynucleotide triphosphates, will not lead to significant shortening of the 3′ strand complementing the adapter primer. The 3′ strand complementing the VNTR primer, however, will be removed by T4 DNA polymerase if the reaction occurs in the presence of the deoxynucleotides that it lacks. Exonuclease digestion by the enzyme will cease when the first deoxynucleotide that is present in the reaction mixture is encountered. The 5′ overhang that is created may be digested with a single strand specific exonuclease or endonuclease, including but not limited to Exonuclease VI , such that all repeat sequence is removed. The illustration depicts a scenario for (AC)n and (GT)n primed amplimers:

[chemical structure]

If a trinucleotide VNTR has been targeted appropriate digestion by T4 DNA poiymerase in the presence of only one deoxynucleotide will be required. For tetranucleotide repeats this method is inappropriate and another should be adopted.

(B) The repeat sequence may be digested with a 5′ to 3′ exonuclease, such as T7 gene 6 exonuclease. Phosphorothioate bonds retard the activity of this enzyme. Four successive bonds are believed to inhibitory. Therefore, if the adapter primer has been synthesised with at least four phosphorothioate bonds at its 5′ end, if not synthesised completely with phosphorothioate bonds, it will be resistant to the 5′ to 3′ exonuclease activity of T7 gene 6 exonuclease. If the VNTR primers are synthesised with four phosphorothioate bonds at their 3′ ends, the action of T7 gene 6 exonuclease will digest the VNTR primer leaving four nucleotides of repeat sequence. The complementary sequence may be digested by a single strand specific exonuclease or endonuclease, including but not limited to Exonuclease I, such that all repeat sequence is removed from the amplimers apart from four nucleotides in each strand. Such a short length of repeat sequence is unlikely to invite the generation of spurious products by non-specific interaction of strand ends during hybridisation.

[chemical structure]

(C) Synthesis of uracil containing VNTR primers, e.g. (GU)nH and (UG)nV, allows the destruction of these primers in the appropriate amplimer pool by the action of Uracil DNA glycosylase. Incubation of the digested amplimers with a single strand specific endonuclease, including but not limited to S1 nuclease, leads to further digestion of the VNTR primers that contains single stranded spaces and ultimately to the removal of the complementary sequence such that all repeat sequence is removed.

[chemical structure]

(D) The generation of amplimer pools with RNA primers based on VNTR sequence, using a DNA polymerase with reverse transcriptase activity, permits the destruction of the VNTR primers by the action of RNAse. The complementary sequence may be removed by a single strand specific exonuclease or endonuclease.

There are several methods by which the digested amplimers may be hybridised to the genomic DNA of one or more individuals to generate en masse and with fidelity the VNTR alleles as they occur in that template. These include (A) hybridisation and polymerisation of the amplimer pools, either separately in succession or together to genomic DNA that may or may not have been fragmented; (B) hybridisation and polymerisation of the amplimers constituting only one flank of each VNTR to genomic DNA that has been fragmented physically, chemically or enzymatically, and then terminated and ligated to an adapter which may or may not be the one used to generate the amplimer pools. In each case, the addition of one of many hybridisation accelerators will enhance the rate of hybridisation. Particularly under stringent conditions of hybridisation the use of such accelerators may be preferable. The number of methods by which hybridisation may be accelerated is vast but includes the incorporation of phenol exclusion, cationic detergents such as cetyl trimethylammonium bromide (CTAB), and volume excluding agents such as dextran sulphate. It should be noted that if CTAB is the chosen hybridisation accelerator the salt concentrations in the hybridisation mixture should be low in order to prevent its precipitation.

(A) Illustration is given for hybridisation of one amplimer pool to genomic DNA to permit the reproduction of VNTR alleles in that genomic template by a DNA polymerase:

[chemical structure]

Hybridisation of the second amplimer pool permits amplification of ail VNTR alleles en masse using the adapter primer:

[chemical structure]

(B) Illustration is given for hybridisation of one amplimer pool to genomic DNA that has been fragmented, terminated and ligated to an adapter that may or may not be the same as that as that present in the amplimer pools:

[chemical structure]

Removal of repeat sequence from the amplimers permits concomitant hybridisation of both amplimer pools to genomic DNA while limiting the possibility for generation of spurious products through non-specific strand association. The generation of spurious products is reduced further by hybridising the amplimers that constitute each flank separately in succession. This allows the introduction of further steps to control non-specific strand association including the removal of non-hybridised strands by incubation with a single strand specific exonuclease or endonuclease between hybridisations. In the preferred technique only one amplimer pool, comprising one flank of each VNTR, is hybridised to terminated and adapter-ligated genomic fragments. As such, this negates any possibility of non-specific association between amplimer strands of different pools. If each amplimer pool is hybridised and polymerised separately in this manner, the products that are generated in each reaction should be identical. Therefore, these products may be combined.

Hybridisation of the amplimers to the pooled genomes of several individuals allows the generation of the VNTR alleles that they contain. If this is performed on the pooled genomes of individuals manifesting a particular trait, and also on those of individuals lacking the trait, the ‘affected’ and ‘wild type’ alleles that are present in those pooled genomes can be synthesised.

It is preferable to select the affected individuals from a defined population such that the same genotype is common to all individuals of a given phenotype. However, even if these individuals are selected from an out-bred population for which there are several genotypes that produce a single phenotype, the alleles that co-segregate with the trait loci will be present at a higher frequency in the pooled genomes of affected individuals than in the reciprocal pooled genomes of wild type individuals. These alleles will be enriched by successive repetitions of mis-match cleavage and amplification. To prevent the allele frequencies from being artificially skewed it is preferable to have a large number of individuals contributing genomic DNA to each pool. This ensures that the allele frequencies in the affected group and wild type group tend to equate to the general population from which they are derived such that disparity in the two is a consequence of linkage disequilibrium with the trait and not another factor. However, if the numbers of affected and wild type individuals is limited the selection of matched sibling pairs, one member of each pair being affected and the other being a wild type individual, will go some distance to balance the allele frequencies of the pooled genomes other than with respect to the particular trait.

### Mis-match discrimination

If the VNTR alleles that are generated from the affected individuals and the wild type individuals are denatured and allowed to re-anneal in separate reactions duplex DNA molecules with or without mis-matches will result. Due to the VNTR-specific flanking sequences and stringent conditions of hybridisation, only alleles that are of the same VNTR will re-anneal. Therefore, duplexes possessing mis-matches contain alleles of the same VNTR that are of unequal size or they contain spurious products of amplification. Alleles of similar size that re-anneal will form perfect duplexes.

The molecules that contain a mis-match may be digested with an enzyme that acts upon single stranded DNA or an enzyme that is able to detect conformational irregularities in DNA. Suitable enzymes include but are not limited to S1 nuclease and T4 endonuclease VII.

[chemical structure]

Of these two enzymes, T4 endonuciease VII has proved to be the most reliable and efficient enzyme in this application and has been found to digest efficiently in a range of DNA polymerase buffers while tolerating carry-over of CTAB from the hybridisation reaction. It cleaves both strands of a mis-match containing molecule leaving staggered ends, each strand being cleaved 3′ with respect to the mis-match.

Cleavage is likely to occur within the repeat sequence creating ends that may interact non-specifically during the subsequent amplification process and resulting in the generation of spurious products. To obviate this problem the repeat sequences may be digested from the cleaved duplexes. This may be achieved in a number of ways, including (A) by the action of a 3′ to 5′ exonuclease including but not limited to Exonuclease III, together with a single strand specific exonuclease or endonuciease, having protected all DNA strands prior to T4 endonuclease VII digestion with protective termini including but not limited to U-thiophosphate groups or a 3′ overhang; (B) by the action of a 5′ to 3′ exonuciease including but not limited to T7 gene 6 exonuclease, together with an exonuclease or endonuclease, having protected all DNA strands prior to T4 endonuclease VlI digestion with protective groups including but not limited to phosphorothioate bonds incorporated in to the adapter primer.

By inclusion of phosphorothioate bonds in the adapter primer the 5′ ends of all molecules containing the adapter primer will be resistant to the 5′ to 3′ exonuclease activity of T7 gene 6 exonuclease. However, the 5′ ends created by T4 endonuclease Vll cleavage will be susceptible to this enzyme.

[chemical structure]

It is possible that some molecules will escape complete cleavage by T4 endonuclease VlI acquiring merely a single stranded nick. However, such nicks are susceptible to digestion by 17 gene 6 exonuclease, though only the nicked strand would be digested if this enzyme was used in concert was a single strand specific exonuclease. On the other hand, a single strand specific endonuclease, including but not limited to S1 nuclease, would cleave the complementary single strand that is exposed by action of T7 gene 6 exonuclease in molecules receiving single stranded nicks such that both strands become disrupted. Thus, enzymes such as S1 nuclease in concert with 17 gene 6 exonuclease would lead to the complete digestion of all T4 endonuclease VII digested molecules irrespective of whether one or both strands was cut.

S1 nuclease has proven successful in this role, being capable of efficient digestion of single stranded DNA under alkaline conditions created by the 7 gene 6 exonuclease buffer. However, some non-specific digestion of DNA may occur with this enzyme. Since those molecules receiving single stranded nicks by the action of T4 endonuclease VlI are likely to be few, it may be preferable to use a single strand specific exonuclease that is less likely to act in this way. Among such enzymes are included Exonuclease I and Exonuclease VII. Molecules that lack a mis-match are resistant to this regime of digestion and may be enriched by amplification. In order to minimise the generation of ‘stutter bands’ that result from strand slippage and polymerase errors during the amplification reaction, the number of cycles of amplification should not exceed that which gives adequate yields of product.

In addition to T7 gene 6 exonuclease, Exonuclease IlIl may act at nicks in DNA molecules. In the absence of phosphorothioate bonds within the adapter primer this enzyme would create long 3′ overhangs in nicked molecules on digestion to completion. Therefore, inclusion of a single strand specific endonuclease or exonuclease that would remove these overhangs would allow the elimination of the cleaved molecule irrespective of whether T4 endonuclease Vil disrupted one or both strands in a mis-match containing duplex. However, in order to obviate the need for the additional step comprising protection of the 3′ ends of all DNA molecules prior to mis-match cleavage the use of T7 gene 6 exonuclease is preferred since protection of the 5′ ends that is required for use of this enzyme is easily achieved by incorporation of phosphorothioate bonds into the adapter primer.

Another method by which cleaved molecules could be removed is by addition of a hapten, including but not limited to biotin-16-dUTP, at the sites of cleavage followed by physical separation of the cleaved molecules by the affinity of the hapten to another chemical. This could be achieved by termination of the 3′ ends of all molecules prior to the mis-match cleavage procedure such that they are inert in the presence of a DNA polymerase. Suitable termini include but are not limited to dideoxynucleotide triphosphates which may be incorporated by a DNA polymerase including but not limited to Terminal deoxynucleotidyl transferase. Subsequent incubation of the cleaved molecules with biotin-16-dUTP in the presence of a DNA polymerase, such as Terminal deoxynucleotidyl transferase, will give rise to biotinylation of only those molecules which lack terminated 3′ ends. Separation of the biotinylated molecules through binding to streptavidin could then follow.

In a similar manner, since molecules cleaved by T4 endonuclease VII have a 3′ overhang these molecules could be removed through capture by single stranded binding proteins or chemicals that possess an affinity for single stranded DNA. It is likely that the overhang created by T4 endonuclease VII will be too small for efficient selection of the cleaved molecules by this method. However, they could be lengthened specifically by incubation with a DNA polymerase, including but not limited to Terminal deoxynucleotidyl transferase in the presence of one or more deoxynucleotide triphosphates, having terminated all 3′ ends of the DNA molecules prior to mis-match cleavage with suitable termini that render them inert in the presence of a DNA polymerase.

Physical separation of DNA molecules is cumbersome and relatively inefficient compared to separation by enzymatic means. Furthermore, the removal of molecules that possess single stranded nicks is likely to be unsuccessful. For these reasons methods of enzymatic differentiation of DNA species is preferred.

Reiteration of several rounds of denaturation, hybridisation and mis-match cleavage successfully eliminates all spurious products of amplification. Furthermore, it reduces to homozygosity all VNTRs such that only the most common allele of each VNTR remains, or it tends to eliminate those VNTRs for which many alleles are present with equal frequency. Rapid transition from the temperature of denaturation to that of annealing is required to prevent preferential annealing of identical sized alleles. This is may occur if the transition from the denaturation temperature to the annealing temperature is protracted. A hybridisation accelerator may be included to enhance the efficiency of hybridisation. This process carried out in parallel for the ‘affected’ VNTR alleles as well as the ‘wild type’ VNTR alleles will tend to achieve identical reduction to homozygosity and the generation of balanced allele frequencies. However, for a number of VNTRs the allele frequencies in the affected and wild type groups at the end of the mis-match cleavage procedure will be significantly different. Providing that the trait of interest is the only feature distinguishing the two groups of individuals from which the VNTRs were derived alleles that are over represented in the affected group relative to the wild type group must co-segregate with that trait. These are markers of the trait and should be selected.

The effect of reiterated mis-match cleavage on the allele frequencies of a VNTR can be illustrated with a basic scenario ignoring the efficiency of digestion, the effects of polymerase errors and the second order kinetics of hybridisation. Consider a VNTR for which three alleles are present as follows:

### STARTING SCENARIO

| Alleles | A | B | C |
|---|---|---|---|
| Allele frequency | 2/4 | ¼ | ¼ |
| Ratio | 2 | 1 | 1 |

If the alleles are denatured and allowed to re-anneal duplex molecules with or without a mismatch will result. The proportion of each allele that forms a perfect duplex will depend on its allele frequency. All mis-match containing molecules theoretically would be susceptible to digestion by T4 endonuclease VlI and would be eliminated. Thus, after the first round of mis-match cleavage the amounts and ratios of each allele remaining would be:

| Alleles | A | B | C |
|---|---|---|---|
| Amount remaining | 4/16 | 1/16 | 1/16 |
| Total remaining | 6/16 | | |
| Ratio | 4 | 1 | 1 |
| Allele frequency | 4/6 | 1/1 | ⅙ |

After a second round of mis-match cleavage the allele frequencies would change further:

| Alleles | A | B | C |
|---|---|---|---|
| Amount remaining | 16/36 | 1/36 | 1/36 |
| Total remaining | 18/36 | | |
| Ratio | 16 | 1 | 1 |
| Allele frequency | 16/18 | 1/18 | 1/18 |

After the 3rd round the theoretical allele frequencies would be as follows:

| Alleles | A | B | C |
|---|---|---|---|
| Amount remaining | 256/324 | 1/324 | 1/324 |
| Total remaining | 258/324 | | |
| Ratio | 256 | 1 | 1 |
| Allele frequency | 256/258 | 1/258 | 1/258 |

Therefore, after two rounds one allele would predominate markedly. After a further round this allele would be present virtually exclusively. The ratio of the total amount of this VNTR remaining, relative to a VNTR for which there was only one allele prior to mis-match cleavage, would be:

$$\frac{6}{16} \times \frac{18}{36} \times \frac{258}{324} : \frac{1}{1} \times \frac{1}{1} \times \frac{1}{1} = \\ \frac{43}{288} : 1$$

In the same way the most common allele of any VNTR will predominate after a sufficient number of rounds of mis-match cleavage. Four rounds may be sufficient to reduce the VNTRs to near homozygosity, but the efficiency of enzyme digestion, the generation of polymerase errors and the kinetics of hybridisation are factors that will influence this. Disparity in the allele frequencies of affected and wild type VNTRs will lead to enrichment of different alleles in each group if the imbalance is sufficiently large. Such alleles are informative for the trait of interest but must be selected from other enriched alleles that may be identical in both the affected and wild type groups if these predominate in the population in general irrespective of the trait.

Further examples of mis-match discrimination under different scenarios is given in the Appendix.

Selection of alleles informative for a trait

Selection of the alleles linked to the trait of interest may be achieved in a number of ways. Disparity in the allele size of each VNTR surviving successive rounds of the mis-match cleavage procedure may be identified by hybridisation of these alleles from each group of individuals to an array of VNTR alleles of known length and spatial separation such that differences can.be detected. Indeed, it may be possible to achieve quantitative hybridisation to an array in a similar manner that generates information regarding allele frequencies in the two groups without need of the mis-match cleavage procedure.

A less elaborate procedure involves the subtraction of the alleles in one group from those in another to identify differences in allele frequencies. However, this method must identify not only a VNTR for which an allele is present in one group but no alleles survive in the other group, but also a VNTR for which the alleles surviving in each group are different since both of these scenarios suggest linkage disequilibrium with the trait of interest. This can be achieved physically, chemically or enzymatically. If enzyme based selection is chosen it is preferable to amplify the alleles that have been enriched by the mis-match cleavage procedure with adapter primers that lack phosphorothioate bonds in order that enzyme digestion can proceed to completion.

A suitable method of enzyme based selection involves the addition of protective termini, including but not limited to a 3′ overhang of at least four nucleotides or an a-thiophosphate linkage, to the surviving alleles of one group of individuals and subtraction with an excess of those surviving from the other group using Exonuclease Ill. Under most circumstances identification is required of any allele surviving from the affected individuals that fails to survive from those individuals lacking that trait. For this, addition of the protective termini should added only to the VNTRs derived from affected individuals. Obviously, the alternative strategy is possible. A 3′ overhang may be created in a number of ways including but not limited to (A) ligation of an adapter, or by (B) non-template addition of nucleotides by a DNA polymerase. Of these, method (B) was found to be the more efficient which may be achieved using an enzyme such as Terminal deoxynucleotidyl transferase. This enzyme may generate a 3′ overhang of several hundred nucleotides on incubation in the presence of a single deoxynucleotide triphosphate. An x-thiophosphate linkage may be incorporated by addition of a protective deoxynucleotide analogue using a DNA polymerase including but not limited to Terminal deoxynucleotidyl transferase. Suitable analogues include (-thio deoxynucleotide triphosphates. Since these analogues may inhibit subsequent digestion or manipulation of the DNA molecules the addition of a 3′ overhang to impart protection is preferred. Another less preferred method of imparting protection to the activity of Exonuclease III is through the action of an exonuclease with 5′ to 3′ activity, including but not limited to T7 gene 6 exonuclease, that may create a 5′ recess in duplex DNA. The appropriate incorporation of phosphorothioate bonds within the adapter primer that is used to amplify the DNA molecules would ensure that digestion by T7 gene 6 exonuclease beyond that required to impart resistance to Exonuclease III is prevented. Similarly, a 5′ recess could be created by incorporation of a uracil rich 5′ end in the adapter primer which could be digested using an enzyme such Uracil DNA glycosylase.

[chemical structure]

The resulting molecules are resistant to Exonuclease III digestion because of the 3′ overhang that is created. Hybridisation to an excess of the surviving wild type alleles ensures heteroduplex formation of all affected alleles providing an allele of the appropriate VNTR survives in the wild type group.

[chemical structure]

If there are no wild type alleles to subtract from those of the affected group homoduplex molecules that possess a 3′ overhang at each end will result (molecule 1). If the surviving aliele of a VNTR differs between the two groups a heteroduplex molecule containing a mis-match will result (molecule 2). Surviving alleles of equal size in the two groups will give rise to heteroduplex molecules without a mis-match (molecule 3). The other species of DNA that will result from the hybridisation include homoduplexes of wild type alleles that may or may not contain a mis-match (molecule 4) and single stranded molecules that fail to hybridise. Digestion of these different types of molecule by an enzyme that acts on single stranded DNA or conformational irregularities in DNA, including but not limited to T4 endonuclease VII, results in cleavage of those duplexes containing a mis-match with the generation of a 3′ overhang at the site of cleavage.

[chemical structure]

The subsequent digestion by Exonuclease III renders single stranded all duplexes or fragments of duplexes that do not possess a 3′ overhang at each end.

[chemical structure]

Since the digestion of susceptible molecules by Exonuclease III tends to go to completion further digestion with a single strand specific exonuclease or endonuclease eliminates all single stranded DNA species and removes the 3′ overhang on the surviving molecules. Therefore, only the target molecules survive digestion. Exonuclease I is suited to this task but often leaves a single nucleotide 3′ overhang that must be removed if blunt end cloning is chosen as the means by which the target molecules are recovered.

[chemical structure]

For the intact homoduplexes the informative allele is present within the homoduplex and may be identified by cloning and sequencing. For T4 endonuclease VII cleaved fragments that have survived digestion by Exonuclease III and Exonuclease 1, the full length VNTRs can be obtained by hybridisation of the fragments to fragmented, terminated adapter-ligated genomic DNA followed by amplification in a similar manner to that previously described. The informative allele may be identified by genotyping the individuals manifesting the trait of interest with respect to these VNTRs using VNTR-specific primers designed from their flanking sequences.

It is obvious that this method of subtraction is equally suited to other alleles besides those of VNTRs that may be generated in a variety of different ways. As such, this method of identifying differences in the composition of DNA pools may be applied more widely for selection of other types of polymorphic sequences as well as other species of DNA that may be present in one pool but absent in the same form in another.

This method is unique in its suitability for investigation of polygenic as well as monogenic hereditary traits. It is likely to make a significant impact in the study of hereditary traits, reducing considerably the difficulty, time and expense that is currently associated with this field of research.

The preferred embodiment

(i) Fragmentation of genomic DNA of an individual of the species under investigation, but not necessarily an individual in that investigation, with a single restriction enzyme.

(ii) Termination of all 3′ ends by Terminal deoxynucleotidyl transferase in the presence of a dideoxynucleotide triphosphate.

(iii) Ligation of the terminated fragments to an adapter by incubation in the presence of T4 DNA ligase, followed by termination of single-stranded nicks.

(iv) Purification of the ligated products from the ddNTPs and amplification in reactions containing:

a) adapter primer and an (AC)nB primer, where B=G+T+C;

b) adapter primer and a (CA)nD primer, where D=G+A+T;

c) adapter primer and a (GT)nH primer, where H=A+T+C;

d) adapter primer and a (TG)nV primer, where V=G+A+C.

The products of amplification result from genomic fragments that successfully ligate to the chosen adapter and contain a VNTR with homology to the chosen primer.

(v) Digestion of the (AC)nB and (CA)nD primed products by T4 DNA polymerase in the presence of dATP and dCTP, followed by Exonuclease VII to remove all VNTR sequences and excess VNTR primer.

(vi) Digestion of the (GT)nH and (TG)nV primed products by T4 DNA polymerase in the presence of dGTP and dTTP, followed by Exonuclease VII to remove all VNTR sequences and excess VNTR primer. Size selection may be performed to obtain products of an optimal range of molecular weights.

(vii) Hybridization of an excess of either the combined (AC)nB and (CA)nD primed products or the combined (GT)nH and (TG)nV primed products with a sufficient amount of genomic DNAs derived from individuals manifesting a particular trait of interest.

(viii) Incubation of the hybridized products with Taq DNA polymerase to achieve strand extension of all annealed 3′ ends.

(ix) Addition of adapter primer and generation of VNTR alleles from the ‘genomic template’ by thermal cycling in the presence of Taq DNA polymerase.

(x) Purification of the generated VNTR alleles followed by strand dissociation and reannealing under stringent conditions.

(xi) Digestion with T4 endonuclease VII of mis-match containing duplex molecules that result from hybridization of VNTR alleles to spurious products of amplification, or hybridization of VNTR alleles that differ among the individuals under investigation manifesting a particular trait of interest.

(xii) Further digestion by T7 gene 6 exonuclease together with S1 nuclease to remove VNTR sequence from cleaved molecules or eliminate them completely.

(xiii) Amplification of the surviving DNA molecules by thermal cycling in the presence of Taq DNA polymerase.

(xiv) Repetition of hybridization, digestion and amplification of the surviving DNA molecules. This enriches the reaction in VNTR alleles that are common to all individuals manifesting the particular trait of interest or those alleles that predominate in such a group and removes any spurious products of amplification.

(xv) Addition of a 3′ overhang to the selected alleles of the group of individuals manifesting a particular trait by incubation with Terminal deoxynucleotidyl transferase in the presence of a dNTP.

(xvi) Hybridization of the selected VNTR alleles of the group of individuals manifesting a particular trait that possess a 3′ overhang to an excess of the VNTR alleles of individuals in which the trait is absent that have been generated from their genomic DNAs in a method bearing similarity, wholly or in part, with (i) to(xiv).

(xvii) Digestion of mis-match containing duplex molecules by T4 endonuclease VII.

(xviii) Further digestion by Exonuclease IlIl to eliminate strands in duplex molecules that lack protection by a 3′ overhang.

(xix) Further digestion, after removal or inactivation of the Exonuclease ll, by Exonuclease I to remove single stranded DNA. This results in elimination of all molecules other than the VNTRs linked to the particular trait. For intact VNTRs the informative allele is present. For cleaved VNTRs that survive digestion by Exonuclease III and Exonuclease I the entire VNTR sequence may be obtained after hybridisation to fragmented, terminated, adapter-ligated genomic DNA and strand extension by Taq DNA polymerase such that VNTR specific primers may be designed from the flanking sequences that allow genotyping of affected individuals to implicate the informative allele linked to the trait.

A second embodiment

(i) VNTR alleles are generated by means other than processes of amplification of fragmented and ligated genomic DNA with adapter primer and VNTR primer, hybridization of the generated products to genomic ‘template’ DNAs of individuals manifesting a particular trait, and generation of the respective VNTR alleles from those template DNAs. These may include but are not limited to:

a) amplification of VNTRs from genomic or synthetic DNA using primers specific to the flanking regions of each VNTR in individual reactions;

b) amplification of VNTRs from genomic or synthetic DNA using a multiplex system, thereby allowing amplification of multiple VNTRs en masse using adapted VNTR specific primers;

c) amplification of VNTRs from genomic or synthetic DNA using an endonuclease that cleaves in or about VNTR sequences such that adapters may be ligated to the digested DNA and used for amplification of the VNTR alleles;

d) generation of a pool of VNTRs from individuals manifesting a particular trait by processes of subtraction with those in which the trait is absent.

(ii) Purification of the generated VNTR alleles followed by strand dissociation and reannealing under stringent conditions.

(iii) Digestion with T4 endonuclease VlI of mis-match containing duplexes that result from hybridization of VNTR alleles to spurious products of amplification, or hybridization of VNTR alleles that differ among the individuals under investigation manifesting a particular trait of interest.

(iv) Incubation of the hybridized alleles in the presence of 17 gene 6 exonuclease and S1 nuclease such that the digested duplex DNA molecules and single stranded DNA species are eliminated.

(v) Enrichment by amplification of mis-match free duplexes that are resistant to digestion.

(vi) Repetition of hybridization, digestion and selection of mis-match free molecules. This enriches the reaction in VNTR alleles that are common to all manifesting the particular trait of interest and removes any spurious products of amplification.

(vii) Hybridization of the selected VNTR alleles, that are common to all individuals manifesting a particular trait, to the VNTR alleles of individuals in which the trait is absent that have been generated from their genomic DNAs in a method bearing similarity, wholly or in part, with (i) to (vi).

(viii) Digestion with T4 endonuclease VlI of mis-match containing duplexes followed by successive incubation with Exonuclease Ill and Exonuclease I.

(ix) Selection from the mixture of those surviving molecules that lack a 5′ overhang. These entire VNTRs or VNTR fragments are linked to the particular trait of interest. The informative allele, with respect to the trait of interest, of the entire VNTRs can be established by sequencing. For the VNTR fragments the full length sequence can be generated by hybridisation to fragmented, terminated and adapter-ligated genomic DNA followed by incubation with Taq DNA polymerase. The informative allele may be established by various methods including but not limited to genotyping individuals manifesting the trait of interest using VNTR-specific primers designed from the flanking sequences.

Those that are skilled in the art will appreciate that there are several methods of differentiating mis-match containing duplexes from those that are free of mis-matches, either in solution or on an array. The methods described in the above embodiments represent only one of these methods.

Those that are skilled in the art will appreciate that the invention is equally well suited any type of VNTR including but not restricted to dinucleotide repeats e.g.(CA)n and (GT)n, trinucleotide repeats e.g.(AAT)n, (AGC)n, (AGG)n, (CAC)n, (CCG)n and (CTT)n, and tetranucleotide repeats e.g.(CCTA)n, (CTGT)n, (CTTT)n.(TAGG)n, (TCTA)n, and (TTCC)n. In addition, the invention may be applied to simple organism microsatellites that include, but are not limited to, (AT), (CC), (CT) and (GA) rich tracts of repetitive motifs.

Those that are skilled in the art will appreciate that polymorphic alleles, other than those of VNTRs, may be used with the invention to produce alleles that are free of spurious products of amplification and are common to all individuals manifesting a particular trait. These polymorphic alleles may be hybridized to a fixed array of all possible alleles, or subset thereof, or to a pool of alleles derived from individuals in which that trait is absent. By mis-match discrimination those alleles linked and informative for a trait can be identified.

Those that are skilled in the art will appreciate that alleles from the genome of a single individual, or more than one individual, of unknown phenotype and genotype may be amplified with fidelity, removing the spurious products of amplification by mis-match discrimination, and hybridized to a fixed array of alleles, or to a pool of alleles in solution, in order assign a genotype or a phenotype to that individual.

Those that are skilled in the art will appreciate that mis-match discrimination may be performed using enzymes or chemicals other T4 endonuclease VII. These alternatives include but are not limited to S1 nuclease, Mung Bean nuclease, mutation detection proteins (e.g. Mut S), osmium tetroxide and hydroxylamine.

Those that are skilled in the art will appreciate that the polymorphic sequences that are amplified are themselves valuable and may be used in protocols other than that which determines co-segregation of VNTRs with a hereditary trait including but not limited to genotyping, mapping, positional cloning, quantification of trait loci, studies of ancestry and evolution, population studies, phylogenetics, and the study in vitro as well as in vivo of VNTRs and the sequences that separate them.

Those that are skilled in the art will appreciate that the invention may be used to identify somatic mutations that are non-hereditary if a VNTR is involved in that mutation.

Those that are skilled in the art will appreciate that the terminated and adapter-ligated genomic fragments may be used to recreate or amplify that region of the genome with sequence homology to any nucleotide sequence known or unknown to which they are hybridised.

Those that are skilled in the art will appreciate that the method represents a means of purifying a consensus sequence from PCR products such that the spurious products of amplification are eliminated.

Those that are skilled in the art will appreciate that the method represents a means of purifying a consensus sequence from any pool of one or more types of DNA molecule.

The invention differs fundamentally from all previous techniques since genomic fragments are generated that do not reflect the polymorphic variation at the locus from which they were derived. Furthermore, these fragments need not be generated from an individual in a particular investigation, but may be from any individual of the appropriate species. However, hybridization of these fragments to genomic ‘template’ DNA of an individual subject to investigation and mis-match discrimination permits amplification, with fidelity, of alleles within that genomic template whilst overcoming the problems of generation of spurious products that are a feature of other PCR-based methods. If the genomic fragments are derived from a single individual the problems of polymorphic variation within the sequences that flank each VNTR are negated because these will be identical for all individuals under investigation. Since the invention preserves each VNTR allele with its flanking sequences, these alleles remain highly informative. In this respect the invention is unique. Furthermore, this novel method of generating VNTRs is rapid, inexpensive, has no requirement for prior knowledge of sequence, and has no requirement for elaborate equipment, it is of immense importance obviating the high investment of time and money that is currently required for isolation of VNTRs. Consequently, the application of technologies dependant on the availability of VNTR in species in which none have been isolated will be possible where previously this was unfeasible. The ability to generate large numbers of VNTRs from all species quickly, efficiently, cheaply and with fidelity is a considerable contribution of the present invention to workers in the to the biomedical field.

In summary, the invention involves a novel method of generating VNTRs encompassing restriction endonuclease digestion of DNA, ligation of the fragments to adapters and, by introduction of a primer with sequence homology to a chosen VNTR, amplifying only those fragments that are flanked by a chosen endonuclease restriction enzyme site and a VNTR. These fragments are not representative of the alleles of each VNTR and need not be generated from any specific individual under investigation. Hybridization of these fragments with genomic DNA of the individuals under investigation recreates the intact VNTR alleles with flanking sequence, as they occur in the genome. This in itself constitutes a major step in the ability of workers in the biomedical fields to generate quickly, efficiently, cheaply and with fidelity VNTRs in all species for purposes reliant on the availability of VNTRs, including but not confined to DNA fingerprinting and linkage analysis. The incorporation of a mis-match discrimination procedure overcomes the problems of miss-priming and generation of spurious products by reaction contamination and subtle variation in reaction conditions, that are to the detriment of all PCR-based technologies, and allows exclusion of alleles that are not common to all individuals under investigation that manifest a particular trait. A second round of mis-match discrimination removes uninformative alleles that are present in the genomes of individuals that do not manifest the trait. This procedure is designated a Total Representation of Alleles that are Informative for a Trait (TRAIT). The invention, therefore, has significant advantages over previous methods, embracing the speed of analysis of AFLP, GMS, RDA and RAPD, and the high polymorphism detection rate of linkage analysis, but negating the need for DNA from closely related individuals and for paternity testing. The invention also overcomes fundamental problems that are a feature of PCR based technologies, including miss-priming and generation of spurious products through reaction contamination and subtle variations in the conditions of reaction. Furthermore, there is no requirement for expensive equipment or elaborate statistical computer software. The analysis will give rise to alleles that are both linked and informative, being present exclusively or at a higher frequency in individuals manifesting the trait of interest but absent or present at a lower frequency in those individuals that lack the trait. In this respect, the invention is unchallenged in its superiority over all other methods.

The invention allows concomitant detection of polymorphisms at multiple loci by simultaneous comparison of simple or complex genomes from multiple individuals and differs fundamentally from all other techniques that have been previously employed. The invention represents a major advance in the ability of workers in the biomedical fields to generate VNTRs from the genomes of any species quickly, efficiently, cheaply and with fidelity in addition to screening complex genomes for polymorphisms co-segregating with hereditary traits. Application of this procedure will therefore facilitate the development of markers for genetic screening for hereditary disease, or advantageous monogenic or polygenic traits in all organisms.

### Examples of How the Invention may be Applied

The following illustrations represent examples of how the invention may be applied without inferring any limitation to scope of the invention or any limitation to the different ways in which the invention may be applied.

### Experimental Data

### EXAMPLE 1

Preparation of amplimers using (CA)₁₃ and (GU)₁₃ primers.

2 μg DNA was completely digested with 3 μl Rsa I in a total volume of 100 μl:

8.5 μl genomic DNA (equivalent to 3 μg DNA)

10 μl 10× reaction buffer

3 μl Rsa I (10u/μl; Promega)

78.5 μl dH₂O

100 μl

The reaction was incubated at 37° C., over night followed by heat inactivated by incubation at 70° C., for 20 minutes. The DNA was separated from the buffer by microconcentration (Microcon-100; Amicon). A volume of 10 μl was recovered.

2nmoles of 48mer and 2nmoles of 12mer oligonucleotides that constitute the adaptor were combined:

15.9 μl 48mer (equivalent to 2 nmoles)

13.7 μl 12mer (equivalent to 2 nmoles)

10 μl 10× ligase buffer (NEB)

48.4,1 dH₂0

88 μl,

The mixture was heated to 50° C., and allowed to cool to 10° C. over 1 hour.

To the 88 μl, of annealed adaptor was added the 10 μl of digested DNA and ligation of the adaptor to the genomic fragments was performed:

88 μl annealed adaptor/ ligase buffer (containing ATP)

10 μl DNA

2 μl T4 DNA ligase (400 NEBu/μl)

100 82 l

The reaction was incubated at 160C over night and then heat inactivated by incubation at 700C for 20 minutes.

The adaptor-ligated DNA fragments were separated from the buffer and non-ligated adaptor by microconcentration (Microcon-100; Amicon). A volume of 12 μl DNA was recovered.

The adaptor-ligated DNA fragments were incubated with Taq DNA polymerase in the presence of dideoxynucleotide triphosphates to prevent 3′ extension of the adaptor and non-ligated DNA in subsequent manipulations:

12 μl microconcentrated DNA

3 μl 10 × NH₄ reaction buffer

1 μl 50 mM MgCl₂

1 μl 10 mM ddATP

1 μl 10 mM ddCTP

1 μl 10 mM ddGTP

1 μl 10 mM ddTTP

1 μl Taq DNA polymerase (5ul/μl; Bioline)

9 μl dH₂O

30 μl

The reaction was incubated at 72° C. for 2 hours.

The adaptor-ligated DNA with terminated 3′ ends was purified by phenol/chloroform extraction and microconcentration. The volume recovered was made up to 40 μl and the concentration of DNA was gauged by gel electrophoresis. A concentration of 75 ng/μl was determined. (CA) primed amplimers and (GU) primed amplimers were generated in separate reactions:

10 μl, 10× NH₄ reaction buffer

8 μl 50 mM MgCl₂

1.5 μl 10 mM dNTPs

1 μl adaptor-ligated DNA with terminated 3′ ends

4 μl (CA) or (GU) primer (25pmol/μl)

73.5 ul dH₂O

98 μl

The reaction was overlaid with mineral oil and heated to 95° C. for 2 minutes, during which time 1 μl Taq DNA polymerase (5u/μl; Bioline) and 2 μl adaptor primer (50pmol/μl) were added.

Thermal cycling was performed as follows: 95° C., for 30 seconds, then 72° C., for 45 seconds for a total of 20 cycles, followed by 72° C., for 5 minutes.

To the 100 μl of (CA) primed products was added Sμl Exonuclease I (10ulμl) to remove the remaining (CA) primer. This reaction was incubated at 37° C., for 30 minutes.

To the 100 μl of (GU) primed products was added 10 μl Uracil-DNA glycosylase (1 u/μl; NEB) to digest all uracil incorporated into the PCR products. This reaction was incubated at 37° C., for 2 hours. 1 μl 10 mM dNTPs was added followed by 2 μl T4 DNA polymerase (5u/μl; Epicentre laboratories) to remove the protruding (CA) strand that complemented the digested (GU) sequence. This reaction was incubated at 37° C., for 5 minutes. Both the pools of amplimers were phenol/chloroform extracted and microconcentrated (Microcon-100; Amicon). For each pool, the volume recovered were made up to 500 μl, of which 5 μl was analysed by spectrophotometry to determine the concentration of DNA.

Equal amounts of (CA) and (GU) primed amplimers were hybridized to genomic ‘template’ DNA of a single individual prior to thermal cycling. In order to gauge the optimal ratio of amplimer to genomic ‘template’ DNA several reactions were performed using various amounts of ‘template’ DNA while keeping the amount of amplimers constant:

| | | | | | | |
|---|---|---|---|---|---|---|
| ′Template′ DNA (ng) | 0 | 0.1 | 1 | 10 | 100 | 1000 |
| Combined amplimers | 1 | 1 | 1 | 1 | 1 | 1 |
| (ng) | | | | | | |
| 5M NaCl (μl) | 0.22 | 0.22 | 0.22 | 0.22 | 0.22 | 0.22 |

| | |
|---|---|
| dH₂O (μl) | To a final volume of 5.55 μl |

Each reaction was overlaid with mineral oil and incubated at 98° C. for 5 minutes, after which the temperature was reduced stepwise to 78° C. over 4 hours.

The following was added to each hybridization:

5 μl 10× NH₄ reaction buffer

4 μl 50 mM MgCl₂

0.75 μl 10 mM dNTPs

0.5 μl adaptor primer (50pmol/μl)

34.2 μl dH₂O

Each reaction was spun briefly in a microfuge. They were heated to 72° C. for 2 minutes and 0.5 μl Taq DNA polymerase (5u/μl;Bioline) was added. The reactions were incubated at 72° C. for a further 10 minutes, after which the temperature was raised to 95° C. for 2 minutes. Thermal cycling was performed as follows: 95° C., for 30 seconds, then 72° C. for 1 minute, for a total of 10 cycles.

For each reaction 10 μl of products amplified for 10 cycles were added to 40 μl of reaction mix and amplified under the same conditions for an additional 22 cycles. 5 μl of the ends products of amplification were run on an agarose gel. The reaction containing 100 ng genomic ‘template’ DNA was found to yield the most products of amplification, equivalent to a ratio of 100:1 by mass of genomic ‘template’ DNA: amplimer.

The invention was validated by cloning the products of amplification. Two colonies of E. coli that had successfully transformed ere cultured, from which plasmids were later harvested. These plasmids ere sequenced and were found to contain VNTR sequences at the multiple cloning sites.

### Further experimental data

For the following experiments canine genomic DNA or cloned VNTR alleles amplified from canine genomic DNA were used. The cloned alleles were ligated into the Smal site of the pUC18 MCS, either side of which plasmid specific primers were designed for subsequent amplification of the plasmid inserts:

[chemical structure]

All reagents were obtained from Amersham Pharmacia Biotech, or its subsidiary companies, unless stated otherwise.

Oligonucteotides were obtained from Genset Corp., France. The VNTR primers (AC) 11B, (CA) 11D, (GT) 11H and (TG)11V comprised eleven repetitions of the sequence shown in brackets followed by a degenerate base were B=C+G+T, D=A+G+T, H=A+C+T, and V=A+C+G.

### EXAMPLE 2

Generation of adapter-ligated, dideoxynucleotide terminated genomic fragments with (a) termination preceding adapter ligation and (b) adapter ligation preceding termination.

(a) 5 μg canine genomic DNA were fragmented with Hae III, the digestion proceeding to completion over 12 hours at 37° C.:

4.4 μl 1.135 μg/μl genomic DNA

10 μl 10× restriction buffer

2 μl 10 u/μl Hae III

84 μl dH₂O

100 μl

Digestion was confirmed by electrophoresis of an aliquot of the reaction on a 1 % agarose gel stained with ethidium bromide.

The DNA was extracted (GFX purification column) and eluted in 50 μl mM Tris pH8.5, of which 30l was incubated with Terminal deoxynucleotidyl transferase for 3 hours at 37° C.:

30 μl DNA

30 μl 5× Terminal deoxynucleotidyl transferase buffer

4.5 μl 10 mM ddGTP

10 μl 9u/μl Terminal deoxynucleotidyl transferase

75.5 μl dH₂O

150 μl

The DNA was separated from low molecular weight solutes by microconcentration (Microcon-30; Amicon) with successive additions of dH₂O between episodes of centrifugation. A volume of 35 μl was recovered.

An adapter was prepared by annealing two oligonucleotides, a 24mer (GsCsAsGsGAGACATCGAAGGTATGAAC, where ‘s’ represents a phosphorothioate bond) and a 12mer (TTCATACCTTCG).

7.6 μl 197 pmol/μl 24mer

9.2 μl 162 pmol/μl 12mer

1.87 μl 10×T4 DNA ligase buffer

18.7 μl

The mixture was heated to 55° C. and allowed to cool to 10° C. over one hour.

The adapter was ligated to the terminated genomic fragments:

35 μl DNA

18.7 μl adapter

4.3 l 10× T4 DNA ligase buffer

1.5 μl 10u/μl T4 DNA ligase

2.5 μl dH₂O

62 μl

The reaction was incubated at 16° C., over night, then heat inactivated at 70° C. for 20 minutes.

The DNA was separated from low molecular weight solutes by microconcentration (Microcon-30; Amicon) with successive additions of dH₂O between episodes of centrifugation. A volume of 54 μl was recovered.

To prevent generation of spurious products through priming from sites of single strand nicks, these were terminated by incubation with Thermo Sequenase:

54 μl DNA

4.4 μl Thermo Sequenase buffer

1.4 μl 10 mM ddATP

1.4 μl 10 mM ddCTP

1.4 μl 10 mM ddGTP

1.4 μl 10 mM ddTTP

0.5 μl 32u/μl Thermo Sequenase

5.5 μl dH₂O

70 μl

The mixture was overlaid with mineral oil and incubated at 74° C., for 2 hours.

The DNA was extracted (GFX purification column) and eluted in 50 μl 5 mM Tris pH 8.5.

(b) 5 μg canine genomic DNA were fragmented with Mbo I, the digestion proceeding to completion at 37° C.:

4.4 μl 1.135μg/μl genomic DNA

10 μl 10× restriction buffer

2.5 μl/μl Mbo I

83 μl dH₂O

100 μl

Digestion was confirmed by electrophoresis of an aliquot of the reaction on a 1% agarose gel stained with ethidium bromide.

Following incubation at 70° C. for 20 minutes the DNA was separated from low molecular weight solutes by microconcentration (Microcon-30; Amicon) with successive additions of dH₂O between episodes of centrifugation. A volume of 32 μl was recovered of which half was ligated to an adapter:

An adapter was prepared by annealing two oligonucleotides, a 24mer (GsCsAsGsGAGACATCGMGGTATGAAC, where ‘s’ represents a phosphorothioate bond) and a 16mer (GATCGTTCATACCTTC):

6.3 μl 197 pmol/μl 24mer

8.5 μl 147 pmol/μl 16mer

1.65 μl 10×T4 DNA ligase buffer

16.5 μl

The mixture was heated to 55° C. and allowed to cool to 10° C. over one hour.

The adapter was ligated to the genomic fragments:

16 μl DNA

16.5 μl adapter

2.4 μl 10× T4 DNA ligase buffer

2 μl 10ul/μl T4 DNA ligase

3.1 μl dH₂O

40 μl

The reaction was incubated at 16° C. over night, then heat inactivated at 70° C. for 20 minutes.

The DNA was separated from low molecular weight solutes by microconcentration (Microcon-30; Amicon) with successive additions of dH₂O between episodes of centrifugation. A volume of 40 μl was recovered.

The adapter-ligated fragments were terminated using Thermo Sequenase:

40 μl DNA

4.4 μl Thermo Sequenase buffer

1.4 μl 10 mM ddGTP

0.5 μl 32u/ul Thermo Sequenase

24 μl dH₂O

70 μl

The reaction was overlaid with mineral oil and incubated at 74° C. for 1 hour. To prevent generation of spurious products through priming from sites of single strand nicks, these were terminated by further incubation with Thermo Sequenase and addition of the remaining ddNTPs:

1.4 μl 10 mM ddATP

1.4 μl 10 mM ddCTP

1.4 μl 10 mM ddTTP

0.3 μl Thermo Sequenase buffer

4.8 μl

The reaction was incubated at 74° C. for a further hour.

The DNA was extracted (GFX purification column) and eluted in 50 μl 5 mM Tris pH 8.5.

Methods (a) and (b) of adapter ligation and termination of the genomic fragments were compared by amplification of the resulting fragments with or without an ‘internal’ primer in reactions comprising the following:

| | | | | | | |
|---|---|---|---|---|---|---|
| 5 | μl | 5 | μl | 5 | μl | 10x Taq PCR buffer |
| 5 | μl | 5 | μl | 5 | μl | 10x dNTPs |
| 1 | μl | 1 | μl | 1 | μl | 25 pmol/μl 24mer |
| 1 | μl | 1 | μl | 0 | μl | 50 pmol/μl (AC)11B |
| 50 | ng | 0 | ng | 50 | ng | GFX extracted DNA |

| | |
|---|---|
| to 50 μl | dH₂O |

Each reaction was overlaid with mineral oil and heated to 95° C. for 2 minutes.

0.5 μl of 5u/μl Taq DNA polymerase was added to each reaction, which was amplified for 25 repetitions of 95° C., for 30 seconds, 65° C. for 30 seconds, 72° C., for 1 minute, followed by a final extension of 72° C., for 5 minutes.

7.5 μl of each reaction was subjected to electrophoresis on a 1.5% agarose gel stained with ethidium bromide. The negative control reactions that lacked DNA generated no product, while those reactions containing all components generated a smear of products of various molecular weights. In contrast, the reactions containing DNA but no internal primer were incapable of generating product. These results confirmed that adapters had been ligated successfully to genomic fragments and all 3′ ends capable of extension in the presence of a DNA polymerase had been terminated. The preferred method was termination prior to ligation since (i) this guaranteed that all fragments successfully ligating were terminated and (ii) the opportunities for inter-fragment ligation were remote.

Amplification of 5′ and 3′ flanking sequences from terminated, adapter-ligated genomic fragments.

Amplification reactions were performed for each VNTR primer containing the following:

| | | |
|---|---|---|
| 5 μl | 5 μl | 10x Taq PCR buffer |
| 5 μl | 5 μl | 10x dNTPs |
| 2 μl | 2 μl | 25 pmol/μl 24mer |
| 2 μl | 2 μl | 25 pmol/μl (AC)11B or (CA)11D or (GT)11H or |
| | | (TG)11V |
| 2 μl | 0 μl | fragmented, terminated, adapter-ligated |
| | | genome (approx. 50 ng/μl) |
| 34 μl | 36 μl | dH₂O |
| 50 μl | 50 μl | |

In addition, a parallel reaction was prepared containing all components except a VNTR primer.

All reactions were overlaid with mineral oil and heated to 95° C., for 2 minutes. 0.5 μl of 5u/μl Taq DNA polymerase was added to each tube and amplification was achieved by thermal cycling for 18 repetitions of 95° C., for 30 seconds, 65° C., for 45 seconds, 72° C., for 45 seconds, followed by a final extension of 5 minutes at 72° C. of each reaction was loaded onto a 1.5% agarose gel stained with ethidium bromide, along with a molecular weight marker. The reactions that contained all components generated a smear of products of ranging from approximately 100 to 500bp, the intensity and distribution of molecular weights being comparable for each reaction. The lanes corresponding to those reactions lacking DNA and the reaction lacking a VNTR primer did not contain any product of amplification.

### EXAMPLE 3

The efficiency of digestion of the repeat sequence from a VNTR primed PCR product by T4 DNA polymerase was assessed.

A cloned VNTR allele was amplified by Taq DNA polymerase and separated from low molecular weight solutes by microconcentration (Microcon-30; Amicon) with successive additions of dH₂O between episodes of centrifugation. A volume of 40 μl was recovered, the concentration of which was judged by agarose gel electrophoresis to be 130ng/μl, approximating to 1.3 pmol/μl.

A 1 .5u/Il dilution of T4 DNA polymerase was prepared with dH₂O. The amplified DNA was digested at a concentration of 0.3 pmol/μl with varying concentrations of T4 DNA polymerase at 12° C.:

| | | |
|---|---|---|
| 1.5 | μl | 10x T4 DNA polymerase buffer |
| 0.75 | μl | 10 mM dATP |
| 0.75 | μl | 10 mM dCTP |
| 3.5 | μl | DNA |
| 0, 0.5, 1, 2, or 4 | μl | 1.5 u/μl T4 DNA polymerase |
| to 15 | μl | dH₂O |

Parallel reactions were prepared that lacked dNTPs. The reactions were incubated at 12° C., for 1 hour, followed by heat inactivation at 70° C., for 20 minutes.

7.5 μl of each reaction were subjected to electrophoresis on a 2.5% agarose gel stained with ethidium bromide. In the absence of dNTPs all DNA was digested with enzyme concentrations exceeding 0.05u/μl. By contrast, there was no discernible loss of DNA in the presence of dNTPs at any concentration of T4 DNA polymerase.

The efficiency of digestion of the repeat sequence from a VNTR primed PCR product by T7 gene 6 exonuclease was assessed.

A cloned VNTR allele was amplified with the plasmid specific sense primer and the (GT)1 1 H primer by Taq DNA polymerase in the presence of [α-33P] dATP. Parallel reactions were performed for primers that contained or lacked a succession of four phosphorothioate bonds. In the primer pair containing phosphorothioate bonds these where located at the 5′ end of the plasmid specific primer and at the 3′ end of the (GT)11 H primer.

The amplified DNA was separated from low molecular weight solutes by microconcentration (Microcon-30; Amicon) with successive additions of dH₂O between episodes of centrifugation. Equal amounts of the amplification reactions were digested by T7 gene 6 exonuclease at 37° C. for 15 and 30 minutes, the concentration of DNA approximating to

0.1 pmol/μl:

| | |
|---|---|
| 3.6 μl | DNA |
| 2 μl | 5x T7 gene 6 exonuclease buffer |
| 1 μl | 10 u/μl T7 gene 6 exonuclease |
| 3.4 μl | dH₂O |
| 10 μl | |

A control reaction was incubated for 15 minutes at 37° C. in the absence of enzyme.

All reactions were denatured at 95° C. for 2 minutes with addition of 5 μl formamide loading dye. 10 μl of each sample was subjected to electrophoresis on an 8% polyacrylamide denaturing gel. An autoradiography film (Biomax MR; Kodak) was exposed to the gel after it had been fixed and dried.

It was found that after 15 minutes of incubation the DNA that lacked phosphorothioate protection had been digested completely. By contrast, the presence of phosphorothioate bonds preserved the DNA, one strand in each molecule becoming shortened by digestion of the enzyme, although some non-specific loss of DNA was seen.

The efficiency and specificity of digestion by T4 endonuclease VlI and S1 nuclease was compared.

Cloned VNTR alleles of the same VNTR that differed in their repeat lengths by 4 nucleotides were amplified separately in the presence of [α-33P] dATP. The products derived from the shorter allele were divided equally between two tubes. To one tube an equal amount of the longer allele was added and the mixture was hybridised by denaturing at 98° C. for 2 minutes and annealing at 75° C., for 150 minutes in 100 mM NaCl and 200 μM CTAB.

The hybridised and non-hybridised pools of DNA were separated from other low molecular weight solutes by microconcentration (Microcon-30; Amicon) with successive additions of dH₂O between episodes of centrifugation.

T4 endonuclease VlI was diluted to 250u/μl in the supplied dilution buffer. Dilutions of S1 nuclease were prepared in dH₂O. Equal amounts of either hybridised DNA or non-hybridised DNA were digested by 50u/μl T4 endonuclease VII in Taq DNA polymerase buffer or by various concentrations of S1 nuclease in the supplied buffer. The S1 nuclease was added to the reactions to give final concentrations of 0.01 u/μl, 0.03u/μl, 0.1u/μl, and 0.3u/μl. In each case a control reaction that lacked enzyme was prepared. The reactions were performed at 37° C., for 30 minutes.

On completion of digestion the reactions were stopped by addition of EDTA and heat inactivation. An amount of formamide loading dye equal to half the reaction volume was added and each reaction was denatured by incubation at 95° C., for 5 minutes. 12 μl of each sample were subjected to electrophoresis on an 8% polyacrylamide denaturing gel. An autoradiography film (Biomax MR; Kodak) was exposed to the fixed and dried gel.

T4 endonuclease VII was found to cleave about half of all DNA derived from hybridisation of approximately equal amounts of two different alleles of the same VNTR, creating a characteristic pattern of cleaved products corresponding to the position of the mis-match within the repeat sequence at the time of cleavage. The DNA derived from the single allele that had not been hybridised and, therefore, comprised mis-match free double stranded DNA was not affected by T4 endonuclease VII. In contrast, the characteristic pattern of cleaved products that was seen with T4 endonuclease VII was not seen in association with S1 nuclease under any of the reaction conditions. As such, T4 endonuclease VII was considered the better of the two enzymes in this application.

Repetition of the T4 endonuclease VII reactions using various concentrations of enzyme for 30 minutes and 1 hour of digestion in 1×Taq PCR buffer, 1×Pfu buffer (Stratagene) and 1×T7 gene 6 exonuclease buffer confirmed that the enzyme digested predictably and reproducibly over a range of reaction conditions, their being no overt non specific digestion of DNA detectable at concentrations up to 200u/μl. The enzyme was found to cleave hybridised molecules containing mismatches of a range of sizes.

The characteristic pattern of cleaved products resulting from a mis-match within a repeat sequence was seen with S1 nuclease only when large amounts of DNA were loaded onto a polyacrylamide gel. This was seen with a four nucleotide mis-match. The ability of S1 nuclease to resolve a two nucleotide mis-match was found to be poor.

The effect of enzyme concentration on the efficiency of cleavage of mis-match containing duplex DNA by T4 endonuclease VII was assessed.

Two cloned VNTR alleles that differed in allele length -by 2 nucleotides were amplified separately using the plasmid specific primers, one of which had been labelled with [γ-33P] ATP using T4 polynucleotide kinase. Each amplified allele was separated from low molecular weight solutes by microconcentration (Microcon-30; Amicon) with successive additions of dH₂O between episodes of centrifugation.

Half of the DNA derived from amplification of the smaller allele was saved. To the remaining half was added approximately an equal amount of amplified DNA of the larger allele. This mixture was denatured at 98° C. for 2 minutes and then annealed at 75° C. for 2 hours in the presence of 100 mM NaCI and 200 μM CTAB, the transition between temperatures occurring rapidly. Separation of the annealed DNA from low molecular weight solutes by microconcentration was repeated.

Serial dilutions of T4 endonuclease VII were prepared in the supplied dilution buffer. The non-denatured smaller allele and the allele mixture that had been denatured and annealed were each digested in Taq DNA polymerase buffer with T4 endonuclease VII at final concentrations of 0u/μl, 50u/μl, 100u/μl and 150u/μl:

| | |
|---|---|
| 6 μl | DNA |
| 1 μl | 10x Taq PCR buffer |
| 3 μl | T4 endonuclease VII |
| 10 μl | |

Incubation at 37° C., was carried out for 30 minutes, after which each reaction was heated to 95° C., for 2 minutes with addition of 5 μl formamide loading dye. 10 μl volumes were subjected to electrophoresis on an 8% polyacrylamide denaturing gel, after which the gel was fixed, dried and exposed to an autoradiography film (Biomax MR; Kodak).

Almost no digestion of the non-denatured smaller allele was detected. The little that was seen was assumed to have occurred as a result of digestion at sites of polymerase error or the annealing of stutter bands during the final cycle of amplification. In the lanes corresponding to the annealed allele mixture the characteristic pattern of digestion was seen to occur in the presence of T4 endonuclease VII. Although the amount of digestion at 100 u/μl appeared to be slightly greater than at 50ul/μl, the degree of digestion at each enzyme concentration was found to be almost uniform.

Similar experiments were performed using various concentrations of T4 endonuclease VII in Pfu buffer (Stratagene) and T7 gene 6 exonuclease buffer. Efficient digestion of mis-match containing DNA was found to occur in both reaction buffers, the degree of digestion maximising at concentrations of T4 endonuclease VII between 50u/μl and 100u/μl. Duplex DNA lacking a mis-match was resistant to T4 endonuclease VII under these conditions.

The efficiency and specificity of S1 nuclease digestion in T7 gene 6 exonuclease buffer was assessed.

A cloned VNTR allele was amplified with the plasmid specific primers, one of which had been labelled with [γ-33P] ATP using T4 polynucleotide kinase. The amplified product was separated from low molecular weight solutes by microconcentration (Microcon-30; Amicon) with successive additions of dH₂O between episodes of centrifugation, The volume of recovered DNA was divided: 30 μl was preserved as double stranded DNA while the remaining 30ytl DNA was rendered single stranded by denaturation at 98° C. for 2 minutes followed by snap cooling on iced water.

Dilutions of S1 nuclease were prepared in dH₂O. Equal amounts of double stranded DNA or single stranded DNA were digested in T7 gene 6 exonuclease buffer at 37° C. for 5 minutes in the presence of Si nuclease at final concentrations of 0u/μl, 0.1 u/μl, 0.3u/μl, 1 u/μl and 3uu/μl. On completion of digestion the reactions were stopped by addition of 500 mM EDTA pH8 to a final concentration of 25 mM.

The reactions were denatured by addition of formamide loading dye and heating to 95° C. for 3 minutes, after which aliquots were subjected to electrophoresis on an 8% polyacrylamide denaturing gel. The gel was fixed, dried, and exposed to an autoradiography film (Biomax MR; Kodak).

It was found that a concentration of 1 ul/μl S1 nuclease in T7 gene 6 exonuclease buffer produced optimal digestion of single stranded DNA, there being no overt loss of double stranded DNA at this concentration.

Assessment of the digestion of DNA by T7 gene 6 exonuclease in concert with S1 nuclease.

For assessment of T7 gene 6 exonuclease and S1 nuclease, DNA was amplified from a cloned VNTR allele using the plasmid specific sense primer with four phosphorothioate bonds at the 5′ end and either the (AC)11 B primer containing four phosphorothioate bonds at the 3′ end or the (AC)11 B primer that lacked such bonds. The amplified products were separated from low molecular weight solutes by microconcentration (Microcon-30; Amicon) with successive additions of dH₂O between episodes of centrifugation. The volumes recovered in each case were measured to be 40 μl. These were found to contain approximately 1.3 pmol/μl and 0.35 pmol/μl for the reactions primed by the VNTR primer with and without phosphorothioate bonds, respectively.

T7 gene 6 exonuclease was diluted to 10 u/μl in dH₂O.

S1 nuclease was diluted to 10 u/μl in dH₂O.

Each amplified product, at a concentration of approximately 0.1 pmol/μl, was digested by T7 gene 6 exonuclease. In addition, the DNA generated with the (AC)1 1 B primer containing phosphorothioate bonds was digested by T7 gene 6 exonuclease in concert with S1 nuclease:

| | | | | with | | |
|---|---|---|---|---|---|---|
| without PT bonds | | | | PT bonds | | with PT bonds |
| 4 | μl | 4 | μl | 4 | μl | 5x T7 gene 6 buffer |
| 5.7 | μl | 1.6 | μl | 1.6 | μl | DNA |

| | | | |
|---|---|---|---|
| 0, 2, 4, 8 μl | 0, 2, 4, 8 μl | 0, 2, 4, 8 μl | 10 u/μl |
| | | | T7 gene 6 |
| | | | exonuclease |

| | | | | | | |
|---|---|---|---|---|---|---|
| 0 | μl | 0 | μl | 2 | μl | 10 u/μl S1 nuclease |
| to 20 | μl | to 20 | μl | to 20 | μl | dH₂O |

Each reaction was incubated at 37° C., for 10 minutes, after which 1 μl 500 mM EDTA pH8 was added to each tube followed by incubation at 70° C., for 20 minutes.

10 μl of each digest was subjected to electrophoresis on a 2.5% agarose gel stained with ethidium bromide. Lanes corresponding to reactions lacking enzyme contained a discrete band of the expected molecular weight. The appearance of a lower molecular weight band, corresponding to single stranded DNA, was seen at a concentration of 1 u/μl T7 gene 6 exonuclease for DNA primed by the (AC)11 B primer that lacked phosphorothioate protection. At concentrations exceeding this virtually all DNA was single stranded. In contrast, DNA protected by phosphorothioate bonds at each end did not appear to alter significantly in molecular weight at any of the concentrations of T7 gene 6 exonuclease, but a decrease in the amount of DNA was evident with increasing concentrations. Similarly, DNA protected at each end was resistant to digestion of T7 gene 6 exonuclease in combination with S1 nuclease. Concentrations of 1 u/μl T7 gene 6 exonuclease with 1u/μl S1 nuclease in T7 gene 6 exonuclease buffer containing approximately 0.1 pmol/μl DNA appeared to give the best results.

The mis-match discrimination procedure was assessed using a model system comprising three alleles of the same VNTR in concert with a single allele of a second VNTR.

A mixture of VNTR alleles was prepared that contained three alleles of the same VNTR, (AC)10, (AC)11, and (AC)18, in a 2:1: 1 ratio respectively. In addition, an amount of the (CA)16 allele of a second VNTR, equal to that of the (AC)11 and (AC)18 alleles, was added to the mixture. Using Pfu DNA polymerase (Stratagene) 1 ng of the mixture was amplified by PCR in a reaction volume of 100 μl containing 60 pmoles of each plasmid specific primer, the sense primer having been labelled with [γ-33P] ATP. Thermal cycling was performed for 17 repetitions of 95° C. for 30s, 65° C. for 30s, 72° C., for 45s, followed by a final extension of 72° C., for 5 minutes.

The amplified DNA was separated from low molecular weight solutes by microconcentration (Microcon-30; Amicon) with addition of dH₂O between episodes of centrifugation. The recovered DNA was denatured at 98° C. for 2 minutes and then annealed at 75° C., for 2 hours in 100 mM NaCl and 200 μM CTAB, the transition between temperatures being rapid.

The hybridised DNA was separated from low molecular weight solutes by microconcentration (Microcon-30; Amicon) with addition of dH₂O between episodes of centrifugation, and digested by T4 endonuclease VII in Taq DNA polymerase buffer containing 50u/μl of the enzyme in a total volume of 36 μl. Digestion proceeded at 37° C., for 1 hour after which the reaction was incubated at 75° C., for 15 minutes.

The digested DNA was separated from low molecular weight solutes by microconcentration (Microcon-30; Amicon) with addition of dH₂O between episodes of centrifugation. Further digestion was performed in a 50 μl reaction containing 1u/μl T7 gene 6 exonuclease and 1u/41 S1 nuclease in T7 gene 6 exonuclease buffer at 37° C., for 10 minutes. The reaction was stopped by addition of 2 μl 500 mM EDTA pH8 and heating to 75° C., for 10 minutes.

Microconcentration was performed (Microcon-30; Amicon) with addition of dH₂O between episodes of centrifugation. A volume of 48 μl was recovered of which 4 μl was amplified by PCR, as before. This was followed by a second round of the mis-match discrimination procedure.

Aliquots of the amplified DNA before and after each round of the mis-match discrimination procedure were subjected to electrophoresis on an 8% polyacrylamide denaturing gel. In addition, for comparison of the molecular weight of each product, the PCR products of each allele amplified in isolation were loaded onto the gel.

It was found that Pfu generated numerous stutter bands in each amplification reaction. The amount of the (AC)10 allele in the mixture prior to mis-match discrimination was approximately twice that of all other alleles. These others were present in approximately equal amounts. After the first round of mis-match discrimination obvious enrichment of the (AC)10 allele was seen. This was enhanced by the second round of mis-match discrimination giving rise to a very strong band corresponding to the (AC)10 allele and marked reduction of the (AC)11 and (AC)18 alleles. Although a band corresponding to the (CA)15 allele of the second VNTR was present after the second round of mis-match discrimination it was not as bright as that of the enriched (AC)10 allele. This was considered to reflect the inequality in the total DNA of each VNTR within the mixture and the consequential relative inefficiency of hybridisation following second order kinetics. This experiment confirmed that mis-match discrimination enriches the allele in a mixture of alleles of the same VNTR that has the highest frequency.

### EXAMPLE 4

The protocol was assessed using the pooled genomes of several dogs.

In the absence of DNA samples from individuals affected and unaffected by a hereditary trait the protocol was validated on a model system designed to mimic a scenario of VNTR linkage disequilibrium that would be expected in the presence of a recessive trait.

A total of 43 dogs were genotyped with respect a VNTR previously isolated in the dog using VNTR specific primers. The VNTR primer pair comprised (CACTTGGGACTTTGGATTGGTCA) sense primer and (GTCTTTGTTTCCATTCTTGCTTGC) antisense primer.

Amplification reactions by PCR were performed in a volume of 10 μl containing 20 ng genomic DNA and 4 pmoles of each VNTR specific primer. In each case the VNTR specific sense primer was labelled and added to an amplification reaction master mix:

| | |
|---|---|
| 1.5 μl | 10x T4 polynucleotide kinase buffer |
| 2.4 μl | 50 pmol/μl VNTR specific sense primer |
| 4.5 μl | [γ-33] ATP |
| 1 μl | 1 in 3 dilution of 30 u/μl T4 polynucleotide kinase |
| 5.6 μl | dH₂O |
| 15 μl | |

The reaction was incubated at 37° C., for 1 hour, then 90° C., for 5 minutes.

The T4 polynucleotide kinase reaction was added to a PCR master mix:

| | | |
|---|---|---|
| 15 | μl | T4 polynucleotide kinase reaction |
| 45 | μl | 10x Taq DNA polymerase buffer |
| 45 | μl | 10x dNTPs |
| 2.4 | μl | 50 pmol/μl VNTR specific antisense primer |
| 4.5 | μl | 5 u/μl Taq DNA polymerase |
| 293 | μl | dH₂O |
| 405 | μl | |

For each dog 1 μl of 20 ng/μl genomic DNA was added to 9 μl of PCR master mix which was overlaid with mineral oil. Each reaction was placed onto a preheated thermal cycler at 95° C. and incubated for 2 minutes. Thermal cycling then followed with 28 repetitions of denaturation at 95° C., for 30s, annealing at 65° C. for 30s, and extension at 72° C. for 30s, followed by a final extension of 72° C. for 5 minutes.

On completion of thermal cycling 541 of formamide loading dye was added to each reaction with denaturation at 90° C. for 3 minutes prior to electrophoresis at 60W on an 8% polyacrylamide denaturing gel. The gel was fixed in 10% methanol/10% glacial acetic acid and dried. An autoradiography film (BioMax MR; Kodak) was exposed to the gel overnight.

The genotype of each dog was scored with respect to the VNTR. Ten dogs were selected to represent the ‘affected pool’ of individuals and ten were selected to represent the ‘wild type pool’. This selection was made in order to achieve a scenario that may mimic a recessive trait:

| Affected | Allele frequency |
|---|---|
| (AC)n | 100% |
| (AC)n + 1 | 0% |
| (AC)n + 2 | 0% |
| (AC)n + 3 | 0% |
| (AC)n + 4 | 0% |
| (AC)n + 5 | 0% |
| (AC)n + 6 | 0% |
| (AC)n + 7 | 0% |

| Wild type | Allele frequency |
|---|---|
| (AC)n | 15% |
| (AC)n + 1 | 0% |
| (AC)n + 2 | 0% |
| (AC)n + 3 | 0% |
| (AC)n + 4 | 35% |
| (AC)n + 5 | 20% |
| (AC)n + 6 | 0% |
| (AC)n + 7 | 30% |

Amplimers were prepared from genomic DNA of a single dog. In a 100 μl volume 5 μg of genomic DNA were digested by 20 units Hae III, the digestion proceeding to completion over 12 hours at 37° C.:

| | | |
|---|---|---|
| 4.4 | μl | 1.135 μg/ul genomic DNA |
| 10 | μl | 10x restriction buffer |
| 2 | μl | 10 u/μl Hae III |
| 84 | μl | dH₂O |
| 100 | μl | |

Digestion was confirmed by electrophoresis of an aliquot of the reaction on a 1% agarose gel stained with ethidium bromide.

The DNA was extracted (GFX purification column) and eluted in 50 μl 5 mM Tris pH8.5, of which approximately 3 μg contained within 30 μl was incubated with Terminal deoxynucleotidyl transferase for 3 hours at 37° C.:

| | | |
|---|---|---|
| 30 | μl | DNA |
| 30 | μl | 5x Terminal deoxynucleotidyl transferase buffer |
| 4.5 | μl | 10 mM ddGTP |
| 10 | μl | 9 u/μl Terminal deoxynucleotidyl transferase |
| 75.5 | μl | dH₂O |
| 150 | μl | |

The DNA was separated from low molecular weight solutes by microconcentration (Microcon-30; Amicon) with successive additions of dH₂O between episodes of centrifugation. A volume of 35 μl was recovered.

An adapter was prepared by annealing two oligonucleotides, a 24mer (GsCsAsGsGAGACATCGAAGGTATGAAC, where ‘s’ represents a phosphorothioate bond) and a 12mer (TTCATACCTTCG):

| | | |
|---|---|---|
| 7.6 | μl | 197 pmol/μl 24 mer |
| 9.2 | μl | 162 pmol/μI 12 mer |
| 1.87 | μl | 10x T4 DNA ligase buffer |
| 18.7 | μl | |

The mixture was heated to 55° C., and allowed to cool to 10° C. over one hour.

The adapter was ligated to the terminated genomic fragments:

| | | |
|---|---|---|
| 35 | μl | DNA |
| 18.7 | μl | adapter |
| 4.3 | μl | 10x T4 DNA ligase buffer |
| 1.5 | μl | 10 u/μl T4 DNA ligase |
| 2.5 | μl | dH₂O |
| 62 | μl | |

The reaction was incubated at 16° C., over night, then heat inactivated at 70° C., for 20 minutes.

The DNA was separated from low molecular weight solutes by microconcentration (Microcon-30; Amicon) with successive additions of dH₂O between episodes of centrifugation. A volume of 54 μl was recovered.

To prevent generation of spurious products through priming from sites of single strand nicks, these were terminated by incubation with Thermo Sequenase:

| | |
|---|---|
| 54 μl | DNA |
| 4.4 μl | Thermo Sequenase buffer |
| 1.4 μl | 10 mM ddATP |
| 1.4 μl | 10 mM ddCTP |
| 1.4 μl | 10 mM ddGTP |
| 1.4 μl | 10 mM ddTTP |
| 0.5 μl | 32 u/μl Thermo Sequenase |
| 5.5 μl | dH₂O |
| 70 μl | |

The mixture was overlaid with mineral oil and incubated at 74° C., for 2 hours.

The DNA was extracted (GFX purification column) and eluted in 50 μl 5 mM Tris pH 8.5.

Amplimers were prepared from this DNA using VNTR primers and the 24mer oligonucleotide contained within the adapter as the adapter primer:

| | |
|---|---|
| 5 μl | 10x Taq DNA polymerase buffer |
| 5 μl | 10x dNTPs |
| 2 μl | 25 pmol/μl adapter primer |
| 2 μl | 25 pmol/μl VNTR primer [(AC)11 B, (CA)11 D, (GT)11 H, |
| | or (TG)11V] |
| 2 μl | terminated, adapter-ligated DNA fragments |
| | (approx. 50 ng/μl) |
| 34 μl | dH₂O |
| 50 μl | |

2 μl terminated, adapter-ligated DNA fragments (approx.

50 ng/μl)

34 μl dH₂O

50 μl

Similar reactions were prepared containing a VNTR primer but in the absence of genomic DNA. In addition, a single reaction was performed containing genomic DNA but in the absence of a VNTR primer. All reactions were overlaid with mineral oil and incubated at 95° C. for 2 minutes. Addition of 0.5 μl of ⁵u/μl Taq DNA polymerase was made to each reaction. Amplification was achieved by thermal cycling for 18 repetitions of 95° C. for 30 s, 65° C. for 45s, 72° C., for 45s, followed by a final extension of 72° C. for 5 minutes.

On completion of amplification 5 μl of each reaction were subjected to electrophoresis with a molecular weight marker on a 1.5% agarose gel stained with ethidium bromide. The presence of amplified products in the lanes representing reactions containing template DNA and a VNTR primer confirmed that ligation of the genomic fragments to adapter sequence had occurred. In each case the appearance of these lanes was similar, there being a smear of amplified products distributed over a range of molecular weights from approximately 100 bp to 500 bp. All other lanes lacked product of amplification. The fact that the reaction containing template DNA but no VNTR primer did not generate product confirmed that the all 3′ ends had been terminated successfully such that chain extension in the presence of Taq DNA polymerase was prevented.

The (AC)11B and (CA)11D primed reactions were combined. Also, the (GT)11H and (TG)11V primed reactions were combined. Both amplimer pools were separated from low molecular weight solutes by microconcentration (Microcon-30; Amicon) with addition of dH₂O between episodes of centrifugation. Quantification by agarose gel electrophoresis of the recovered DNA suggested that each contained approximately 35 ng/μl amplimer DNA.

The repeat sequences were removed from the pooled (AC)11B and (CA)11D primed products using T4 DNA polymerase and Exonuclease VII:

| | |
|---|---|
| 14 μl | 35 ng/μl (AC)11B/(CA)11D primed amplimer DNA |
| 2 μl | 10x T4 DNA polymerase buffer |
| 1 μl | 10 mM dATP |
| 1 μl | 10 mM dCTP |
| 2 μl | 1 in 4 dilution of 4 u/μl T4 DNA polymerase |
| 20 μl | |

The reaction was incubated at 12° C., for 1 hour then inactivated at 70° C., for 20 minutes.

To the reaction was added 1 μl of 10u/μl Exonuclease VII with incubation at 37° C., for 30 minutes followed by 70° C., for 20 minutes.

The designated affected and wild type DNA pools were prepared by combining equal amounts of genomic DNA, quantified by spectrophotometry, of the selected dogs. These were phenol/chloroform extracted and microconcentrated (Microcon; Amicon) with addition of dH₂O between episodes of centrifugation.

Each pool of genomic DNA was digested by Hae III, terminated using Terminal deoxynucleotidyl transferase, and ligated to the adapter in a manner similar to that previously described. Complete termination of all 3′ ends was confirmed by PCR with the adapter primer. The DNA pools were quantified by agarose gel electrophoresis and were found to contain approximately equal concentrations.

In a minimal volume 2.5 μl of the 35 ng/μl (AC)/(CA) primed amplimer pool, digested with T4 DNA polymerase and Exonuclease VII, were hybridised in 0.6M NaCl to approximately 300 ng of the ‘affected’ genomic DNA pool that had been fragmented, terminated, and ligated to the adapter. This was achieved by denaturing the mixture under mineral oil at 98° C. for 3 minutes, followed by a stepwise reduction in the temperature from 80° C. to 70° C. over ten hours and sustaining the final temperature for a further 10 hours. The wild type pool was hybridised in a similar manner in parallel.

To each hybridisation were added:

| | |
|---|---|
| 20 μl | 10x Taq DNA polymerase buffer |
| 20 μl | 10x dNTPs |
| 160 μl | dH₂O |
| 200 μl | |

In each case the total volume containing the hybridised DNA was divided between two reaction tubes. Under mineral oil each volume was heated to 750C. 1 l of 5u/μl Taq DNA polymerase was added to each tube followed by incubation at 72° C. for 10 minutes. The reactions were denatured at 95° C. for 3 minutes and 4 μl of 25 pmol/μl adapter primer were added. Amplification of the hybridised DNA was achieved by thermal cycling for 30 repetitions of 95° C. for 30s, 65° C. for 30s, 72° C. for 90s, followed by a final extension of 72° C. for 5 minutes.

The reactions containing affected DNA were pooled, as were the reactions containing wild type DNA, and 8 μl of 10u/μl Exonuclease I were added to each 20041 volume of amplified DNA. The reactions were incubated at 37° C. for 15 minutes.

For each reaction the DNA was separated from low molecular weight solutes (Microcon-30; Amicon) with addition of dH₂O between episodes of centrifugation. In each case a volume of 10 μl was recovered. The alleles contained within each sample were denatured and allowed to anneal by incubation under mineral oil at 98° C. for 5 minutes followed by a rapid reduction in temperature to 750C. At 75° C. 2M NaCl and 10 mM CTAB were added to give final concentrations of 50 mM and 500 μM, respectively. The hybridisation reactions were incubated at 75° C. for a further 16 hours.

To each hybridisation reaction was added 150 μl of 5 mM Tris pH 8.5. The diluted hybridisation reactions were then separated from low molecular weight solutes (Microcon-30; Amicon) with addition of dH₂O between episodes of centrifugation. These were judged to contain approximately 10 pmoles DNA. Digestion by T4 endonuclease VII at a concentration of 50u/μl in Taq DNA polymerase buffer was performed in a volume of 100 μl. The digestion proceeded at 37° C. for 30 minutes prior to incubation at 65° C. for 15 minutes.

Each digest was separated from low molecular weight solutes (Microcon-30; Amicon) with addition of dH₂O between episodes of centrifugation. The recovered volume in each case was divided between three tubes, each being digested either by 0.5u/μl Exonuclease I in 1×Taq DNA polymerase buffer, 1 u/μl T7 gene 6 exonuclease followed after heat inactivation at 70° C. for 10 minutes by 0.5u/lp Exonuclease I in 1×T7 gene 6 exonuclease buffer, or 1u/μl T7 gene 6 exonuclease together with 1 u/μl S1 nuclease in 1×T7 gene 6 exonuclease buffer. The concentration of DNA in each reaction was approximately 0.pmol/μl contained within a 30 μl volume. The Exonuclease I reactions were performed at 37° C. for 15 minutes prior to heat inactivation at 70° C. for 10 minutes. The reactions containing T7 gene 6 exonuclease with or without S1 nuclease were performed at 37° C., for 10 minutes. On completion of each regime of digestion the DNA was extracted (GFX purification column) and eluted in 50 μl dH₂O.

Three quarters of each of the extracted DNA samples was amplified by PCR with Taq DNA polymerase

| | | |
|---|---|---|
| 37.5 | μl | digested DNA |
| 15 | μl | 10x Taq DNA polymerase buffer |
| 15 | μl | 10x dNTPs |
| 6 | μl | 25 pmol/μl adapter primer |
| 76.5 | μl | dH₂O |
| 150 | μl | |

The reactions were divided into 75 μl aliquots and overlaid with mineral oil to which were added 0.75 μl of 5u/μl Taq DNA polymerase after incubation at 95° C., for 2 minutes. Amplification was achieved by thermal cycling for 25 repetitions of 95° C. for 30s, 65° C. for 30s, 72° C. for 90s, followed by a final extension of 72° C. for 5 minutes.

To each 150 μl of amplified DNA were added 6 μl 10 u/μl Exonuclease I. The reactions were incubated at 37° C. for 15 minutes.

The DNA in each case was separated from low molecular weight solutes (Microcon-30; Amicon) with addition of dH₂O between episodes of centrifugation. Repetition of hybridisation in 50 mM NaCl and 500M CTAB followed by each regime of digestion was repeated, followed by amplification of the resulting DNA by PCR with Taq DNA polymerase, as above.

Aliquots of each of the amplified samples were subjected to electrophoresis on a 1.5% agarose gel stained with ethidium bromide with a molecular weight marker. The amplified products in the lanes corresponding to DNA digested by T4 endonuclease VII followed by Exonuclease I were of high molecular weight smearing towards the well. In contrast, the lanes corresponding to amplified product that had been digested by either 17 gene 6 exonuclease followed by Exonuclease I or T7 gene 6 exonuclease concomitantly with S1 nuclease contained products ranging in molecular weights from approximately 200 bp to 750 bp. The distribution of molecular weights in each case was similar. No smearing towards the well was seen suggesting that the spurious products of amplification that were seen in the absence of T7 gene 6 exonuclease were eliminated by the presence of this enzyme. As such, T7 gene 6 exonuclease was considered an essential component of the mis-match discrimination regime for removal of repeat sequences from T4 endonuclease VII cleaved molecules that would otherwise cross-hybridise and produce spurious DNA molecules.

To each of the 150 μvolumes of amplified DNA resulting from the second round of mis-match discrimination were add 6 μl of 10u/μl Exonuclease I and the reactions were digested at 37° C. for 15 minutes.

The DNA in each case was separated from low molecular weight solutes (Microcon-30; Amicon) with addition of dH₂O between episodes of centrifugation.

For each of the reactions corresponding to the ‘affected’ dogs amplification was performed by PCR with Taq DNA polymerase using the VNTR specific primers in a volume of 50 μl containing approximately 25 ng DNA. Amplification by 28 repetitions of thermal cycling was performed after which 5 μl aliquots and a molecular weight marker were loaded onto a 2% agarose gel stained with ethidium bromide.

For the lanes corresponding to digestion by T4 endonuclease VII and Exonuclease I the product of the expected molecular weight was very faint. In addition a large amount of spurious product in the vicinity of the wells was seen. For all other lanes no high molecular weight products were seen. Furthermore, the amplified products were seen clearly as a discrete band of the expected molecular weights of approximately 130 bp.

The products of amplification corresponding to digestion by T4 endonuclease VII and Exonuclease I were discarded. The remaining reactions were amplified further using the VNTR specific primers, one of which was labelled with [7-33P] ATP using T4 polynucleotide kinase. Amplification reactions were performed by PCR using Taq DNA polymerase in volumes of 20 μl containing 10 pmoles of each primer for 35 repetition of thermal cycling. In addition, reactions were performed in the same manner containing 40 ng of the pooled ‘affected’ and pooled ‘wild type’ DNA. After addition of 10 μl of formamide loading dye to each sample the amplified products were denatured at 90° C. for 3 minutes. 6pd aliquots of the mixture were subjected to electrophoresis on an 8% polyacrylamide denaturing gel. The gel was fixed and dried and exposed to an autoradiography film.

It was found that product was visible for DNA amplified from affected DNA following the second round of mis-match discrimination. This was seen in both the lanes corresponding to digestion by T7 gene 6 exonuclease followed by Exonuclease I and those corresponding to digestion by T7 gene 6 exonuclease concomitantly with S1 nuclease. In each case the product resembled that resulting from amplification of the pooled affected DNA that had not been subjected to mis-match cleavage. In the case of wild type DNA amplified after the second round of mis-match discrimination no products were discernible.

This experiment confirmed that VNTRs are reproduced with fidelity from the pooled genomes of several individuals, the alleles in each case being preserved, and mis-match discrimination serves to eliminate spurious products of amplification and enrich the VNTR allele of the highest frequency. Although no products were visible for DNA derived from the wild type DNA, it may be that products would become visible with higher loading of DNA on the polyacrylamide gel. As such, further repetition of the mis-match discrimination procedure would be necessary to reduce to near homozygosity the alleles in both DNA pools such that final selection of the informative allele could be achieved.

### EXAMPLE 5

Demonstration of the resistance to Exonuclease III of DNA with a 3′ overhang derived by ligation to an adapter.

A cloned VNTR allele was amplified by Taq DNA polymerase. The amplified DNA was separated from low molecular weight solutes by microconcentration (Microcon-30; Amicon) with successive additions of dH₂O between episodes of centrifugation.

The volume recovered was measured at 44 μl, the concentration of which was determined by agarose gel electrophoresis to be 160 ng/μl, approximating to 1.6 pmol/μl.

The amplified DNA was blunted by T4 DNA polymerase digestion:

| | |
|---|---|
| 42 μl | DNA |
| 3.25 μl | 10 mM dATP |
| 3.25 μl | 10 mM dCTP |
| 3.25 μl | 10 mM dGTP |
| 3.25 μl | 10 mM dTTP |
| 13 μl | 10x T4 DNA polymerase buffer |
| 3.25 μl | 4 u/μl T4 DNA polymerase |
| 59 μl | dH₂O |
| 130 μl | |

The reaction was incubated at 12° C., for 30 minutes, then heat inactivated at 70° C. for 20 minutes. The DNA was separated from low molecular weight solutes by microconcentration (Microcon-30; Amicon) with successive additions of dH₂O between episodes of centrifugation. A volume of 30 μl was recovered.

1600 pmoles of a 21mer oligonucleotide (CTCGCMGGATGGGATGCTCG) were phosphorylated with T4 polynucleotide-kinase diluted to 10 u/μl in the supplied dilution buffer:

| | |
|---|---|
| 3.19 μl | 21 mer oligonucleotide |
| 1.5 μl | 10x T4 DNA ligase buffer |
| 1 μl | 10 u/μl T4 polynucleotide kinase |
| 9.3 μl | dH₂O |
| 15 μl | |

The reaction was incubated at 37° C., for 30 minutes, then heat inactivated at 90° C., for 10 minutes. To the kinase reaction was added 1600 pmoles of a 12mer oligonucieotide (CATCCTTGCGAG). Annealing of the oligos to form an adapter was achieved by heating to 55° C., and allowing the mixture to cool to 10° C., over a period of 1 hour.

Half of the DNA blunted by T4 DNA polymerase was saved. To the annealed adapter was added the remaining 151 μl of blunted DNA such that the adapter was in a 50 fold excess:

| | |
|---|---|
| 15 μl | blunted DNA |
| 16.2 μl | annealed adapter |
| 1.9 μl | 10x T4 DNA ligase buffer |
| 1 μl | 10 u/μl DNA ligase |
| 34 μl | |

The ligation reaction was incubated over night at 16° C.

The ligation was heat inactivated at 70° C. for 20 minutes and the DNA was separated from low molecular weight solutes by microconcentration (Microcon-30; Amicon) with successive additions of dH₂O between episodes of centrifugation.

The volume recovered was measured to be 36 μl .

The ligated DNA and 15 μl of non-ligated DNA that had been saved were both made to approximately 0.75 pmoles/μl by addition of dH₂O. Each was digested by Exonuclease μlμl at a final concentration of DNA approximating to 0.2 pmol/μl:

| | |
|---|---|
| 10.7 μl | DNA |
| 4 μl | 10x Exonuclease III buffer |
| 1 μl | 200 u/μl Exonuclease III |
| 24.3 μl | dH₂O |
| 40 μl | |

The reaction was incubated 37° C., for 5 minutes then heat inactivated at 70° C. for 20 minutes.

Approximately 2 pmoles of each digest were loaded onto a 2% agarose gel stained with ethidium bromide. All non-ligated DNA was digested to completion by Exonuclease III such that none was detectable on the agarose gel. In contrast, although some digestion had occurred, much of the ligated DNA was found to be resistant to digestion. That which had been digested was assumed to have failed to ligate to the phosphorylated adapter. This experiment confirmed that ligation of an adapter is one method by which DNA molecules may become resistant to Exonuclease III digestion, those molecules lacking an adapter being digested to completion by this enzyme.

Selection of unique sequences in a pool of DNA hybridised to a second pool of DNA using Exonuclease III.

Two cloned VNTR alleles that differed in their repeat lengths by four nucleotides were amplified by PCR using Taq DNA polymerase. The amplified DNAs were separated from low molecular weight solutes by microconcentration (Microcon-30; Amicon) with successive additions of dH₂O between episodes of centrifugation and the resulting concentrations of DNA were determined by agarose gel electrophoresis.

To a portion of the amplified products of the smaller allele was added a 3′ overhang by incubation with Terminal deoxynucleotidyl transferase:

| | | |
|---|---|---|
| 12.5 | μl | 120 ng/μl DNA (approx. 1.2 pmol/μl) |
| 15 | μl | 5x Terminal deoxynucleotidyl transferase buffer |
| 1.125 | μl | 10 mM dATP |
| 3.3 | μl | 9 u/μl Terminal deoxynucleotidyl transferase |
| 43 | μl | dH₂O |
| 75 | μl | |

The reaction was incubated at 37° C., for 1 hour after which the DNA was extracted (GFX purification column).

To 450 ng of the allele possessing a 3′ overhang was added:

(i) 4.5kg of the same allele that lacked a 3′ overhang;

(ii) 4.5kg of the larger allele that lacked a 3′ overhang.

In each case, the total volume was minimised by microconcentration (Microcon-30; Amicon). These mixtures were denatured at 98° C., for 3 minutes and annealed at 75° C., for 2 hours in the presence of 0.2M NaCl and 100 M CTAB.

To each hybridisation reaction were added:

| | |
|---|---|
| 10 μl | 10x Taq DNA polymerase buffer |
| 10 μl | 500 u/μl T4 endonuclease VII |
| 80 μl | dH₂O |
| 100 μl | |

The reactions were incubate at 37° C., for 45 minutes, then inactivated at 70° C., for 15 minutes.

The DNAs were separated from low molecular weight solutes by microconcentration (Microcon-30; Amicon) with successive additions of dH₂O between episodes of centrifugation. In each case a volume of approximately 40 μl was recovered which was diluted in a reaction mixture containing 5u/μl Exonuclease III:

| | | |
|---|---|---|
| 40 | μl | DNA |
| 15 | μl | 10x Exonuclease III buffer |
| 3.75 | μl | 200 u/μl Exonuclease III |
| 91 | μl | dH₂O |
| 150 | μl | |

The reactions were incubated at 37° C., for 5 minutes, after which they were microconcentrated (Microcon-30; Amicon). The entire recovered volumes were subjected to electrophoresis on a 1.5% agarose gel stained with ethidium bromide. In addition, a molecular weight marker, 400 ng of the small allele without a 3′ overhang, and 400 ng of the smaller allele that possessed an overhang were loaded on to the gel.

The size of the smaller amplified allele was confirmed to be approximately 150 bp by comparison to the molecular weight marker. After incubation with Terminal deoxynucleotidyl transferase the apparent size of this amplified allele had increased. A smear of products distributed over a range of sizes corresponding to between 400 bp and 750 bp of double stranded DNA was seen, though the majority of DNA was confined to an ill-defined band midway between these. In the lane containing hybridised alleles of different sizes that had been digested, a band corresponding to approximately 300 bp of double stranded DNA was seen against a back ground smear of products. This band was considered to be the result of enzymatic cleavage of the mis-match containing DNA duplexes, where as the back ground smear was considered to be single stranded DNA resulting from Exonuclease III digestion of molecules lacking the protection of a 3′ overhang. In the lane that contained hybridised alleles of the same size two ill-defined bands were visible against a background smear of products. The brightest band was of an appearance similar to that of the smaller allele following its incubation with Terminal deoxynucleotidyl transferase and was considered to represent the remaining single stranded DNA from heteroduplex molecules digested by Exonuclease III. The fainter band was considered to the result of enzymatic cleavage of molecules possessing polymerase errors. As before, the background smear was considered to be due to single stranded DNA of molecules lacking a 3′ overhang that had resulted from digestion by Exonuclease III. This experiment suggests that an allele possessing a 3′ overhang entering into a heteroduplex with an allele of a different repeat length is digested by T4 endonuclease VII and Exonuclease III such that a fragment of the heterodupiex may be selected.

### Appendix

Consider a scenario that may typify a rare recessive trait. The affected group of individuals are homozygous for the same allele. In the wild type group, this allele has a relatively low frequency.

| | |
|---|---|
| Affected | Wild Type |

| Alleles | A | B | C | D | A | B | C | D |
|---|---|---|---|---|---|---|---|---|
| Starting scenario | | | | | | | | |
| Allele frequencies | 1.0 | 0.0 | 0.0 | 0.0 | 0.15 | 0.35 | 0.2 | 0.3 |
| Allele ratios | 1 | 0 | 0 | 0 | 3 | 7 | 4 | 6 |
| After 1^{st} Round | | | | | | | | |
| Amount remaining | 1.000 | 0.000 | 0.000 | 0.000 | 0.023 | 0.123 | 0.040 | 0.090 |
| Total remaining | 1.0 | | | | 0.276 | | | |
| Allele ratios | 1 | 0 | 0 | 0 | 23 | 123 | 40 | 90 |
| Allele frequencies | 1.000 | 0.000 | 0.000 | 0.000 | 0.083 | 0.446 | 0.145 | 0.326 |
| After 2^{nd} Round | | | | | | | | |
| Amount remaining | 1.0 | 0.0 | 0.0 | 0.0 | 0.006 | 0.199 | 0.021 | 0.106 |
| Total remaining | 1.0 | | | | 0.332 | | | |
| Allele ratios | 1 | 0 | 0 | 0 | 6 | 199 | 21 | 106 |
| Allele frequencies | 1.0 | 0.0 | 0.0 | 0.0 | 0.018 | 0.599 | 0.063 | 0.319 |
| After 3^{rd} Round | | | | | | | | |
| Amount remaining | 1.0 | 0.0 | 0.0 | 0.0 | 0.000 | 0.359 | 0.004 | 0.102 |
| Total remaining | 1.0 | | | | 0.465 | | | |
| Allele ratios | 1 | 0 | 0 | 0 | 0 | 359 | 4 | 102 |
| Allele frequencies | 1.0 | 0.0 | 0.0 | 0.0 | 0.000 | 0.772 | 0.008 | 0.219 |
| After 4^{th} Round | | | | | | | | |
| Amount remaining | 1.0 | 0.0 | 0.0 | 0.0 | 0.000 | 0.596 | 0.000 | 0.010 |
| Total remaining | 1.0 | | | | 0.606 | | | |
| Allele ratios | 1 | 0 | 0 | 0 | 0 | 596 | 0 | 10 |
| Allele frequencies | 1.0 | 0.0 | 0.0 | 0.0 | 0.000 | 0.983 | 0.000 | 0.017 |

| Comparison of the | 1 × 1 × 1 × 1 = 1 0.276 × 0.332 × 0.465 × 0.606 = 0.026 | |
|---|---|---|
| ratios of remaining | 38.5:1 | |
| alleles | | |
| | all of which is A | none of which is A |

Therefore, even if an large excess of wild type DNA is hybridised to the affected DNA that survives the mis-match discrimination procedure it is extremely likely that the aliele present in the affected group will be recovered.

Consider another scenario in which one allele is present in the affected group of individuals at a frequency greater than that of the wild type group.

| | Affected | | | | | Wild Type | | | | |
|---|---|---|---|---|---|---|---|---|---|---|
| Alleles | A | B | C | D | E | A | B | C | D | E |
| Starting scenario | | | | | | | | | | |
| Allele frequencies | 0.050 | 0.100 | 0.000 | 0.150 | 0.700 | 0.250 | 0.200 | 0.150 | 0.250 | 0.150 |
| Allele ratios | 1 | 2 | 0 | 3 | 14 | 5 | 4 | 3 | 5 | 3 |
| After 1^{st} Round | | | | | | | | | | |
| Amount remaining | 0.003 | 0.010 | 0.000 | 0.023 | 0.490 | 0.063 | 0.040 | 0.023 | 0.063 | 0.023 |
| Total remaining | 0.526 | | | | | 0.212 | | | | |
| Allele ratios | 3 | 10 | 0 | 23 | 490 | 63 | 40 | 23 | 63 | 23 |
| Allele frequencies | 0.006 | 0.019 | 0.000 | 0.044 | 0.932 | 0.297 | 0.189 | 0.108 | 0.297 | 0.108 |
| After 2^{nd} Round | | | | | | | | | | |
| Amount remaining | 0.000 | 0.000 | 0.000 | 0.002 | 0.869 | 0.088 | 0.036 | 0.012 | 0.088 | 0.012 |
| Total remaining | 0.871 | | | | | 0.236 | | | | |
| Allele ratios | 0 | 0 | 0 | 2 | 869 | 22 | 9 | 3 | 22 | 3 |
| Allele frequencies | 0.000 | 0.000 | 0.000 | 0.002 | 0.998 | 0.373 | 0.153 | 0.051 | 0.373 | 0.051 |
| After 3^{rd} Round | | | | | | | | | | |
| Amount remaining | 0.000 | 0.000 | 0.000 | 0.000 | 0.996 | 0.139 | 0.023 | 0.003 | 0.139 | 0.003 |
| Total remaining | 0.996 | | | | | 0.307 | | | | |
| Allele ratios | 0 | 0 | 0 | 0 | 1 | 139 | 23 | 3 | 139 | 3 |
| Allele frequencies | 0.000 | 0.000 | 0.000 | 0.000 | 1.000 | 0.453 | 0.075 | 0.010 | 0.453 | 0.010 |
| After 4^{th} Round | | | | | | | | | | |
| Amount remaining | 0.000 | 0.000 | 0.000 | 0.000 | 1.000 | 0.205 | 0.006 | 0.000 | 0.205 | 0.000 |
| Total remaining | 1.0 | | | | | 0.416 | | | | |
| Allele ratios | 0 | 0 | 0 | 0 | 1 | 205 | 6 | 0 | 205 | 0 |
| Allele frequencies | 0.000 | 0.000 | 0.000 | 0.000 | 1.000 | 0.493 | 0.014 | 0.000 | 0.493 | 0.000 |

| Comparison of the | 0.526 × 0.871 × 0.996 × 1 = 0.456 0.212 × 0.236 × 0.307 × 0.416 = 0.006 | |
|---|---|---|
| ratios of remaining | 76:1 | |
| alleles | | |
| | all of which is E | none of which is E |

Therefore, even if an large excess of wild type DNA is hybridised to the affected DNA that survives the mis-match discrimination procedure it is extremely likely that allele E present in the affected group will be recovered.

### References

Bruford M W, and Wayne R K (1993) Microsatellites and their application to population genetic studies. Current Opinion in Genetics and Development. 3; 939-943.

Callen D F, Thompson A D, Phillips H A, Richards R I, Mulley J C, and Sutherland GR (1993) Incidence and origin of ‘null’alleles in the (AC)n microsatellite markers. Am J Hum Genet. 52; 922-927.

Murphy G (1993) Generation of a nested set of deletions using Exonuclease III. Methods in Molecular Biology. 23; 51-59.

Clark D and Steven Henikoff (1994) Ordered deletions using Exonuclease III. Methods in Molecular Biology. 31; 47-55.

Cooney A J (1997) Use of T4 DNA polymerase to create cohesive termini in PCR products for subcloning and site-directed mutagenesis. BioTechniques. 24; 30-34.

Epplen J T, Buitkamp J, Bocker T and Epplen C (1995) Indirect gene diagnoses for complex (multifactorial) disease- a review. Gene 159; 49-55.

Esteban J A, Salas M, and Blanco L (1992) Activation of S1 nuclease at neutral pH. Nucleic Acids Research. 20; (18): 4932.

Hearne C M, Ghosh S, and Todd J A (1992) Microsatellites for linkage analysis of genetic traits. Trends Genet. 8; (8): 288-294.

Karp A, Seberg O and Buiafti M (1996) Molecular Techniques in the Assessment of Botanical Diversity. Annals of Botany 78; 143-149.

Lisitsyn N A (1995) Representational difference analysis: finding the differences between genomes. Trends in Genetics, 11; 303-307.

Lu J, Knox M R, Ambrose M J and Brown J K M (1996) Comparative analysis of genetic diversity in pea assessed by RFLP- and PRC-based methods. Theoretical and Applied Genetics 93;1103-1111.

Mackill DJ, Zhang Z, Redona E D and Colowit P M (1996) Level of polymorphism and genetic mapping of AFLP markers in rice. Genome 39; 969-977.

Molyneux K, and Batt R M (1994) Five polymorphic canine microsatellites. Animal Genetics. 25; 379.

Murphy G (1993) Generation of a nested set of deletions using Exonuclease III. Methods in Molecular Biology. 23; 51-59.

Nelson SF, McCusker JH, Sander MA Kee Y, Modrich P, and Brown PO (1993) Genomic mis-match scanning: a new approach to genetic linkage mapping. Nature Genetics 4; 11-18.

Nikiforov T T, Rendle R B, Kotewicz M L, and Rogers Y (1994) Use of phosphorothioate primers and exonuclease hydrolysis for the preparation of single-stranded PCR products and their detection by solid phase hybridisation. PCR Methods and Applications. 3; 285-291.

Powell W, Morgante M, Andre C, Hanafey M, Vogel J, Tingey S and Rafalski A (1996) The Comparison of RFLP, RAPD, AFLP and SSR (microsatellite) markers for germplasm analysis. Molecular Breeding 2; 225-238.

Vos P, Hogers R, Bleeker M, Reijans M, van de Lee T, Hornes M, Frijters A, Pot J, Peleman J, Kuiper M and Zabeau M (1995) AFLP: a new technique for DNA fingerprinting. Nucleic Acids Research. 23; 4407-4414.

1. A method of making a mixture of VNTR alleles and their flanking regions of the genomic DNA of one or more members of a species of interest, which method comprises the steps of:
a) dividing genomic DNA of the species of interest into fragments,
b) ligating to each end of each fragment an adaptor thereby forming a mixture of adaptor-terminated fragments in which each 3′-end is blocked to prevent enzymatic chain extension,
c) using a portion of the mixture of adaptor-terminated fragments as templates with an adaptor primer and a VNTR primer to create a mixture of 5′-flanking VNTR amplimers,
d) using a portion of the mixture of adaptor-terminated fragments as templates with an adaptor primer and a VNTR antisense primer to create a mixture of 3′-flanking VNTR amplimers,
e) and using genomic DNA of the one or more members of the species of interest as template with the mixture of 5′-flanking VNTR amplimers and/or the mixture of 3′-flanking VNTR amplimers as primers to make the desired mixture of VNTR alleles and their flanking regions.

2. The method of claim 1, wherein step b) is performed by terminating each 3′-end of each fragment to prevent enzymatic chain extension, and ligating each 5′-end of each fragment to an adaptor, thereby forming a mixture of adaptor terminated fragments.

3. The method of claim 1 or claim 2, wherein in step c) the VNTR repeat sequences are removed from the 5′- flanking VNTR amplimers, and in step d) the VNTR repeat sequences are removed from the 3′- flanking VNTR amplimers.

4. The method of any one of claims 1 to 3, wherein in step c) and/or d) the adaptor or primer used contains at least one phosphorothioate bond.

5. The method of any one of claims 1 to 4, wherein step e) is performed using as primers, either successively or together, both the mixture of 5′- flanking VNTR amplimers and the mixture of 3′- flanking VNTR amplimers.

6. The method of any one of claims 1 to 5, wherein there is used in step e) genomic DNA of one or more members of the species of interest which manifest a trait of interest, whereby the resulting mixture of VNTR alleles and their flanking sequences is representative of those which manifest the trait of interest.

7. The method of claim 6 wherein in a step f) the strands of the mixture of VNTR alleles and their flanking regions are separated and then re-annealed and any mismatches are separated and discarded.

8. The method of claim 7, wherein step f) is repeated to recover a single VNTR allele and its flanking regions.

9. The method of any one of claims 6 to 8, wherein at least one VNTR allele and its flanking sequences representative of those which manifest the trait of interest, is hybridised with a mixture of VNTR alleles and their flanking sequences representative of those which do not manifest the trait of interest, and at least one match and/or at least one mis-match is selected to provide at least one VNTR allele or fragment thereof which is characteristic of the trait of interest.

10. The method of claim 9, wherein the at least one VNTR allele and its flanking sequences representative of those which manifest the trait of interest, is provided with 3′-overlapping ends.

11. A portion of genomic DNA of one or more members of a species of interest, said portion consisting essentially of a representative mixture of alleles of a chosen VNTR sequence and their flanking regions on both sides.

12. The portion as claimed in claim 11, wherein the mixture of alleles is representative of those which manifest a trait of interest.

13. The portion as claimed in claim 11 or claim 12, wherein each member of the mixture has an adaptor at each of its 3′-end and its 5′-end.

14. A portion of genomic DNA of one or more members of a species of interest, said portion consisting essentially of a single VNTR allele and its flanking regions and an adaptor at each of its 3′-end and its 5′-end, said allele being characteristic of those which manifest a trait of interest.

15. A portion of genomic DNA of a species of interest, said portion consisting essentially of a representative mixture of 3′-flanking regions of a chosen VNTR sequence, each member of the mixture carrying an adaptor at its 3′-end, and a representative mixture of 5′-flanking regions of a chosen VNTR sequence, each member of the mixture carrying an adaptor at its 5′-end.

16. A method of treating nucleic acids which consist essentially of a mixture of polymorphic alleles, the mixture being representative of those which manifest a trait of interest, which method comprises separating and then re-annealing strands of the mixture, and separating and discarding any mis-matches.

17. The method of claim 16, wherein the mixture of polymorphic alleles is a mixture of alleles of a chosen VNTR sequence and their flanking regions.

18. The method of claim 17, wherein the method is repeated to recover a single VNTR allele and its flanking regions.

19. The method of any one of claims 16 to 18, wherein at least one VNTR allele and its flanking sequence representative of those which manifest the trait of interest, is hybridised with a mixture of VNTR alleles and their flanking sequences representative of those which do not manifest the trait of interest, and at least one match and/or at least one mis-match is selected to provide at least one VNTR allele or fragment thereof which is characteristic of the trait of interest.

20. The method of claim 19, wherein the at least one VNTR allele and its flanking sequence representative of those which manifest the trait of interest, is provided with 3′-overlapping ends.

21. A method of making a mixture of amplimers which method comprises the steps of:
a) dividing genomic DNA of one or more members of a species of interest into fragments,
b) ligating to each end of each fragment an adaptor thereby forming a mixture of adaptor-terminated fragments in which each 3′-end is blocked to prevent enzymatic chain extension, and
c) using a portion of the mixture of adaptor-terminated fragments as templates with an adaptor primer and a VNTR primer to create a mixture of 5′-flanking VNTR amplimers, and/or
d) using a portion of the mixture of adaptor-terminated fragments as templates with an adaptor primer and a VNTR antisense primer to create a mixture of 3′-flanking VNTR amplimers.

22. A method of identifying an allele which is linked to a trait of interest, which method comprises incubating together under hybridisation conditions: at least one molecule of nucleic acid containing a polymorphic allele and its flanking sequences representative of those which manifest the trait of interest; and a mixture of molecules of nucleic acid which contain polymorphic alleles and their flanking sequences representative of those which do not manifest the trait of interest; and selecting at least one match and/or at least one mis-match to provide at least one allele or fragment thereof which is linked to the trait of interest.

23. The method of claim 22, wherein the alleles are VNTR alleles.

24. The method of claim 22 or claim 23, wherein the at least one allele and its flanking sequences representative of those which manifest the trait of interest, is provided with 3′- overlapping ends.

25. Use of the portion of genomic DNA as claimed in claim 14 in a diagnostic assay.

26. The method of any one of claims 1 to 10 or 16 to 20, wherein the VNTR allele and its flanking regions, or the mixture of VNTR alleles and their flanking regions, is analysed by being applied under hybridisation conditions to an array of immobilised VNTR alleles and/or their flanking regions.

27. A kit comprising protocols and reagents for performing the method of any one of claims 1 to 10, 16 to 24 or 26.
